(19) United States
(12) Patent Application Publication
(10) Pub. No.: US 2024/0009108 A1
(43) Pub. Date: Jan. 11, 2024

(54) COSMETIC COMPOSITION, USE OF A COSMETIC COMPOSITION AND PROCESS OF MANUFACTURING A COSMETIC COMPOSITION

(71) Applicant: L'OREAL, Paris (FR)

(72) Inventors: Wagner Gomes PEREIRA, Rio de Janeiro (BR); Pedro Tupinambá Werneck BARROSO, Rio de Janeiro (BR)

(21) Appl. No.: 18/005,781

(22) PCT Filed: Jul. 2, 2020
(86) PCT No.: PCT/BR2020/050242

§ 371 Date: Jan. 17, 2023

(51) Int. Cl. A61K 8/88; A61Q 19/00; A61K 8/92; A61K 8/37; A61K 8/368; A61K 8/25; A61K 8/34

(52) U.S. Cl. CPC A61K 8/88; A61Q 19/00; A61K 8/922; A61K 8/37; A61K 8/368; A61K 8/25; A61K 8/342; A61K 2800/10

(57) ABSTRACT

The present disclosure is directed to new cosmetic compositions comprising (a) at least a polymer, (b) at least a polyamide, (c) at least a filler, (d) at least a fatty compound and (e) at least a nonionic surfactant. The composition of the present invention is stable over the time, also presents a high level of UV-protection in order to protect the skin from the damages of the sun, easy application, good spreadability, very pleasant sensorial, skincare signature, dry touch, mate effect, soft focus effect and light sensorial feeling.

### FIELD OF THE DISCLOSURE

The present disclosure is directed to new cosmetic compositions comprising (a) at least a polymer, (b) at least a polyamide, (c) at least a filler, (d) at least a fatty compound and (e) at least a nonionic surfactant. Preferably, the cosmetic composition of the present disclosure comprises UV filters. The cosmetic composition of the present invention is also related to a process of manufacturing a cosmetic composition and to a use of a cosmetic composition.

### BACKGROUND

Nowadays, the skin care is getting more and more important in the consumer's routine, that desires products that provide multiple benefits, such as hydration, anti-aging effect, controlling of the oiliness, correction of imperfections, sun protection, and makeup effect.

Therefore, it is increasingly desired to have stable compositions with an improved sensorial, which are capable to satisfy the consumer's needs with respect to the type of products mentioned above.

A crucial challenge to develop a cosmetic composition encompassing all the desired attributes mentioned above, is the stabilization of such cosmetic composition, due to the great amount of necessary ingredients to achieve the best performance.

The present disclosure surprisingly developed a new cosmetic composition comprising (a) at least a polymer, (b) at least a polyamide, (c) at least a filler, (d) at least a fatty compound and (e) at least a nonionic surfactant, which provides the desired attributes.

Preferably, the cosmetic composition of the present disclosure further comprises UV filters. In this case, there is an additional challenge when formulating a cosmetic composition with sunscreen protection, since it is necessary to bear in mind that the degree of UV protection afforded by a cosmetic sunscreen composition is directly related to the amount and type of UV filters contained therein. The higher the amount of UV filters, the greater the degree of UV protection (UVA/UVB), and the more difficult to have a stable composition.

The cosmetic compositions with sunscreen protection must provide good protection against the sun, a measure of which is the Sun Protection Factor (SPF) value, yet have satisfactory sensory perception, such as a smooth but not greasy feel upon application. However, this combination of properties has been difficult to achieve, particularly because many active sunscreen compounds themselves have an oily or greasy feel, and increasing their content tends to cause the final product to suffer from that effect.

Also, most organic sunscreen filters are oil-like and/or oil-soluble materials. High levels of sunscreen filters in cosmetic composition with sunscreen protection render the products less appealing for their greasy skin feel, stickiness, long drying time, and leave shiny residue on the skin after application.

Additionally, an important problem of cosmetic compositions with sunscreen protection is that, due to the great amount of sunscreen filters associated with great amount of pigments and further additional ingredients to ensure the good sensorial in the emulsion, it tends to be unstable.

The pigments are raw materials that are used in cosmetic compositions with sunscreen protection and/or makeup compositions for delivering the attribute of uniform the skin tone (appearance of the skin) or coloring the skin. However, such association of pigments to a cosmetic composition, also results in a high complexity challenge to stabilize the emulsion.

Cosmetic compositions with sunscreen protection are quite often in the form of an emulsion of oil-in-water type (O/W) (i.e. a cosmetically acceptable support consisting of a continuous aqueous dispersing phase and of a discontinuous oily dispersed phase) or of the water-in-oil type (W/O) (i.e. a cosmetically acceptable support consisting of a continuous oily dispersing phase and of a discontinuous aqueous dispersed phase) which contains, in varying concentrations, one or more conventional lipophilic and/or hydrophilic organic screening agents (for example the ones which are capable of selectively absorbing harmful UV rays).

It is known that W/O emulsions or inverted emulsions tend to present sticky and unpleasant feeling if not properly prepared, using specific components, especially when formulating a cosmetic composition.

There are several types of emulsions depending on how the oil and water phases are located in the disperse system.

Simple emulsions are labeled as oil-in-water (O/W) when they exhibit oil drops dispersed in an aqueous phase, or water-in-oil (W/O) if water drops is dispersed in an oily phase, also called inverted emulsions. While multiple or double emulsions are symbolized by W1/O/W2 or O1/W/O2. The present disclosure is related to W/O emulsion where dispersed water is contained in a continuous external oil phase.

It is very hard to stabilize inverted emulsions especially with great amounts of active agents, especially with high SPF, where high amount of filters is used. Also, due to the high amount of filters, it is hard to achieve good sensorial, mainly because oil is in the external phase, so the oil phase sensorial is very well perceived in the application.

However, the present disclosure developed an especial combination of fillers, filters (that includes aqueous phase filters, thus helping to reduce filters in the oily phase), and isododecane, a volatile compound that gives a unique sensorial (good spreadability and light sensory) to the formula, even comprising high amounts of UV filters.

The challenge of formulating a cosmetic composition in a W/O form, having high amounts of UV filters, pigments and further additional ingredients associated with the unique sensorial described above, is that such composition tends to be unstable and ineffective.

Similar challenges are present when formulating makeup compositions and skin care compositions, due to chemical nature of the ingredients and their amounts in order to achieve the desired properties.

Therefore, a stable cosmetic composition is desired, associated with improved properties such as easy application, smoothness, good spreadability, less shine, which does not melt at high temperature on the face, having an imperceptible and dry touch, giving the ideal balance between hydration and oil control, which uniform the skin tone or cover imperfections, and which may also provide sunscreen protection.

Thus, the inventors succeeded to overcome the problems of the state of the art and surprisingly revealed a stable cosmetic composition, in the form of a W/O emulsion, comprising (a) at least a polymer, (b) at least a polyamide, (c) at least a filler, (d) at least a fatty compound and (e) at least a nonionic surfactant, presenting a very pleasant sensorial, skincare signature, dry touch, mate effect, pores less evident with a soft focus effect and light sensorial, as well as capable of reducing sebum and easy to apply.

Additionally, when comprising UV filters, the cosmetic composition of the present application provides high UV sunscreen protection.

### SUMMARY

The present disclosure is directed to new cosmetic compositions comprising (a) at least a polymer, (b) at least a polyamide, (c) at least a filler, (d) at least a fatty compound and (e) at least a nonionic surfactant.

Preferably, the cosmetic composition of the present disclosure further comprises UV filters.

The composition of the present invention is stable over the time, easy application, good spreadability, very pleasant sensorial, skin care signature, dry touch, mate effect, pores less evident with soft focus effect and light sensorial as well as capable to reduce sebum. When comprising the UV filters, the cosmetic composition provides high protection to the skin against the damages of the sun.

### DETAILED DESCRIPTION

In an embodiment, the cosmetic composition of the present invention comprises:
(a) at least a polymer, selected from water-soluble or water dispersible AMPS copolymers;
(b) at least a polyamide, selected from the group of aliphatic polyamides;
(c) at least a filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch, and mixtures thereof;
(d) at least a fatty compound, selected from carnauba wax and isononyl isononanoate, and mixtures thereof;
(e) at least a nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants;

In a preferred embodiment, the amount of at least a polymer in the cosmetic composition of the present invention is ranging from about 0.5% to about 10% by weight and preferably from about 1.5% to about 8% by weight, more preferably from about 1.5% to about 6% by weight, including all ranges and sub-ranges there between, based on the total weight of the composition.

In a preferred embodiment, the amount of at least a polyamide in the cosmetic composition of the present invention is ranging from about 1% to about 10% by weight and preferably from about 1.5% to about 8% by weight, more preferably from about 1.5% to about 6% by weight, including all ranges and sub-ranges there between, based on the total weight of the composition.

In a preferred embodiment, the polyamide is selected from polyamide-4, polyamide-6, polyamide-8, polyamide-11, polyamide-12, polyamide-4,6, polyamide-6,6, polyamide-6, 9, and polyamide-6,10, polyamide-6,12, and mixtures thereof.

In a preferred embodiment, the amount of at least a filler in the cosmetic composition of the present invention is ranging from about 1% to about 15% by weight and preferably from about 1.5% to about 10% by weight, more preferably from about 1.5% to about 8% by weight, including all ranges and sub-ranges there between, based on the total weight of the composition.

In a preferred embodiment, the amount of at least a fatty compound in the cosmetic composition of the present invention is ranging from about 1% to about 12% by weight and preferably from about 1.5% to about 10% by weight, more preferably from about 1.5% to about 8% by weight, including all ranges and sub-ranges there between, based on the total weight of the composition.

In a preferred embodiment, the amount of at least a nonionic surfactant in the cosmetic composition of the present invention is ranging from about 1% to about 10% by weight and preferably from about 1.5% to about 8% by weight, more preferably from about 1.5% to about 6% by weight, including all ranges and sub-ranges there between, based on the total weight of the composition.

In another preferred embodiment, the cosmetic composition of the present invention further comprises UV filters and/or pigments.

The cosmetic composition according to the invention presents not only a dry touch and a matte effect but, also leaving the pores less evident with a soft effect and with a light sensorial, even at high concentrations of fillers and/or filters.

In a preferred embodiment, the cosmetic composition presents good stability even when combining high concentrations of ingredients.

Also, in another preferred embodiment, the cosmetic composition presents good stability even when combining high concentrations of fillers and UV filters.

The cosmetic composition of the present invention achieves a very pleasant sensorial, with a skincare signature, dry touch, matte effect, soft focus and light feeling, as well as SPF protection.

In a preferred embodiment, the cosmetic composition of the invention is in the form of an inverted emulsion (W/O or water-in-oil).

In another preferred embodiment, the cosmetic composition of the present invention is related to a cosmetic sunscreen composition that presents a SPF of 30, 50, 60, 70, 90 or 99.

The pH of the cosmetic composition according to the present invention is between 4 and 7.

In a further embodiment, the cosmetic composition of the invention is used in a skin care composition with or without SPF.

In a further embodiment, the cosmetic composition of the present invention can also be used as a makeup composition.

The composition of the present invention is stable over the time, easy application, good spreadability, very pleasant sensorial, skin care signature, dry touch, mate effect, pores less evident with soft focus effect and light sensorial as well as capable to reduce sebum. When comprising the UV filters, the cosmetic composition provides high protection to the skin against the damages of the sun.

Also, the composition of the present invention is stable over the time and presents enhanced emulsion stability.

In another preferred embodiment, the present invention is related to the use of a cosmetic composition for manufacturing a product for preventing sunburn, which can be used as sunscreen daily product, a makeup product or a skin care product.

In a further preferred embodiment, the present invention is related to the use of a cosmetic composition for the manufacturing of a product for makeup the skin.

In a further preferred embodiment, the present invention is related to the use of a cosmetic composition for the manufacturing of a product for skin care.

In another preferred embodiment, the cosmetic composition of the present invention comprises:
(a) from 0.5 to 10% of at least one polymer, selected from a water-soluble or water dispersible AMPS copolymers;
(b) from 1% to 10% of at least a polyamide, selected from the group of aliphatic polyamides
(c) from 1% to 15% of at least one filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch, mixtures thereof;
(d) from 1% to 12% of at least a fatty compound, selected from carnauba wax and isononyl isononanoate and mixtures thereof;
(e) from 1% to 10% of at least one nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants.

In another preferred embodiment the cosmetic composition of the present invention comprises:
(a) from 1 to 6% of at least one polymer, selected from a water-soluble or water dispersible AMPS copolymers;
(b) from 1% to 6% of at least a polyamide, selected from the group of polyamide-4, polyamide-6, polyamide-8, polyamide-11, polyamide-12, polyamide-4,6, polyamide-6,6, polyamide-6, 9, and polyamide-6,10, polyamide-6,12, and mixtures thereof;
(c) from 1.5% to 8% of at least one filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch, and mixtures thereof;
(d) from 1.5% to 10% of at least a fatty compound, selected from carnauba wax, isononyl isononanoate and mixtures thereof;
(e) from 1.5% to 6% of at least one nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants.

In another preferred embodiment, the cosmetic composition of the present invention comprises:
(a) from 1 to 6% of at least one polymer, selected from a water-soluble or water dispersible AMPS copolymers;
(b) from 1% to 6% of at least a polyamide, selected from the group of polyamide-4, polyamide-6, polyamide-8, polyamide-11, polyamide-12, polyamide-4,6, polyamide-6,6, polyamide-6, 9, and polyamide-6,10, polyamide-6,12, and mixtures thereof;
(c) from 1.5% to 8% of at least one filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch, and mixtures thereof;
(d) from 1.5% to 10% of at least a fatty compound, selected from carnauba wax, isononyl isononanoate and mixtures thereof;
(e) from 1.5% to 6% of at least one nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants; and
(f) from 10 to 40% of UV filters.

The process of manufacturing the cosmetic composition of the present invention comprises the following steps:
a) Heating the oily phase containing fatty compounds, emollients and polymer until 75° C.,
a1) when present in the cosmetic composition, adding and heating the UV filters and pigments in step a);
b) Heating the water phase containing polymer and preservatives until 70° C.,
b1) when present in the composition, adding and heating UV filters in step b);
c) Adding the step (a) into step (b) homogenizing the mixture;
d) Adding the fillers and solvent below 45° C. homogenizing the mixture.

Terms

As used herein, the expression “at least” means one or more and thus includes individual components as well as mixtures/combinations.

Other than in the operating examples, or where otherwise indicated, all numbers expressing quantities of ingredients and/or process conditions are to be understood as being modified in all instances by the term “about,” meaning within +/−5% of the indicated number.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “water-in-oil emulsion” means any macroscopically homogeneous, kinetically stable composition comprising at least two mutually immiscible phases; one being the dispersing continuous oily phase and the other being the aqueous phase dispersed in the said continuous oily phase in the form of droplets. The two phases are kinetically stabilized by at least one emulsifying system generally comprising at least one emulsifying surfactant.

Emulsions are distinguished as being of the oil-in-water type, known as “direct emulsions”, consisting of an aqueous dispersing continuous phase and of an oily dispersed discontinuous phase, and emulsions of the water-in-oil type, known as “inverse emulsions”, consisting of an oily dispersing continuous phase and of an aqueous dispersed discontinuous phase. There are also multiple emulsions, for instance water-in-oil-in-water or oil-in-water-in-oil emulsions.

The term “emulsifying system” refers to any compound or mixture of compounds that is capable of increasing the kinetic stability of an emulsion. These compounds are generally amphiphilic and are surfactants characterized by their more or less hydrophilic or more or less lipophilic nature which will determine their ability to stabilize direct emulsions or inverse emulsions. They are especially classified by their HLB according to the calculation method of W. C. Griffin in the document “Classification of Surface Active Agents by HLB, Journal of the Society of Cosmetic Chemists 1 (1949) 311” and in the document “Calculation of HLB of Non Ionic Surfactants, Journal of the Society of Cosmetic Chemists 5 (1954) 249”. The calculation of the HLB according to this calculation method is performed according to the equation:

HLB=20×Mh/M

where Mh is the molar mass of the hydrophilic part of the surfactant and M is the total molecular mass of the molecule.

The term “emulsifying polymer” means any polymer with emulsifying properties thus making it possible to increase the kinetic stability of an emulsion. This polymer is generally amphiphilic and is a surfactant characterized by its more or less hydrophilic or more or less lipophilic nature which will determine its ability to stabilize direct emulsions or inverse emulsions. It is classified especially by its HLB value according to the method indicated above.

For the purposes of the invention, the term “polymer” means a compound containing at least two repeating units, preferably at least three repeating units and better still ten repeating units.

Polymers

The suitable polymers of the present invention are selected from rheology modifier polymers and anionic polymers which may be water-soluble or water-dispersible at a pH of 7 and at room temperature (25° C.). According to the present invention, the suitable polymers of the present invention could be as follows.

The rheology modifier polymers are pre-neutralized and preferably selected from taurate polymers. Such polymers comprise an ionic monomer portion, 2-acrylamido-2-methylpropane sulfonic acid (AMPS), as well as a further, less polar monomer portion (vinylpyrrolidone or beheneth-25 methacrylate). These polymers are used as thickener and as stabilizer for oil-in-water emulsions and form extremely stable emulsions already at low concentrations. In particular, these polymers can be used in conjunction with almost any oil phase, comprising silicone oils, hydrocarbons/waxes and ester oils.

As water-soluble or water-dispersible AMPS copolymers in accordance with the invention, examples that may be mentioned include:
crosslinked acrylamide/sodium acrylamido-2-methylpropanesulfonate copolymers, such as that used in the commercial product Sepigel 305 (CTFA name: Polyacrylamide/C₁₃-C₁₄ Isoparaffin/Laureth-7) or that used in the commercial product sold under the name Simulgel 600 (CTFA name: Acrylamide/Sodium Acryloyldimethyltaurate/Isohexadecane/Poly sorbate-80) by the company SEPPIC;
copolymers of AMPS® and of vinylpyrrolidone or vinylformamide, such as that used in the commercial product sold under the name Aristoflex AVC® by the company Clariant (CTFA name: Ammonium acryloyldimethyltaurateNP copolymer) but neutralized with sodium hydroxide or potassium hydroxide;
copolymers of AMPS® and of sodium acrylate, for instance the AMPS/sodium acrylate copolymer, such as that used in the commercial product sold under the name Simulgel EGO by the company SEPPIC or under the trade name Sepinov EM (CTFA name: Hydroxyethyl acrylate/sodium acryloyldimethyl taurate copolymer);
copolymers of AMPS® and of hydroxyethyl acrylate, for instance the AMPS®/hydroxyethyl acrylate copolymer, such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name Sodium acrylamido-2-methylpropanesulfonate/hydroxyethyl acrylate copolymer, such as the commercial product Sepinov EMT 10 (INCI name: Hydroxyethyl acrylate/sodium acryloyldimethyl taurate copolymer).

Preferably, the product sold under the name Sodium acrylamido-2-methylpropanesulfonate/hydroxyethyl acrylate copolymer, such as the commercial product Sepinov EMT 10 (INCI name: Hydroxyethyl acrylate/sodium acryloyldimethyl taurate copolymer) is used as water-soluble or water-dispersible AMPS copolymers in accordance with the invention.

Examples of taurate polymers are Acrylates/Vinyl Isodecanoate Crosspolymer (Stabylen 30 from 3V), Acrylates/C10-30 Alkyl Acrylate Crosspolymer (Pemulen TR1 and TR2), Carbomers (Aqua SF-1), Ammonium AcryloyldimethyltaurateNP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), Non-ionic thickener, (Aculyn 46 from Rohm and Haas), or mixtures thereof.

Anionic polymers may be polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself.

The anionic polymers may comprise at least one hydrophilic unit of olefinic unsaturated carboxylic acid type, and at least one hydrophobic unit exclusively of (C₁₀-C₃₀)alkyl ester of unsaturated carboxylic acid type.

In certain exemplary and non-limiting embodiments, the copolymers are chosen from the copolymers resulting from the polymerization of:
(1) at least one monomer of formula (I):

CH₂═CH(R₁)COOH (I)

wherein R₁ is chosen from H or CH₃ or C₂H₅, providing acrylic acid, methacrylic acid, or ethacrylic acid monomers, and
(2) at least one monomer of (C₁₀-C₃₀)alkyl ester of unsaturated carboxylic acid type corresponding to the monomer of formula (II):

CH₂═CH(R₂)COOR₃ (II)

Non-limiting examples of (C₁₀-C₃₀)alkyl esters of unsaturated carboxylic acids are for example chosen from lauryl acrylate, stearyl acrylate, decyl acrylate, isodecyl acrylate, dodecyl acrylate and the corresponding methacrylates, such as lauryl methacrylate, stearyl methacrylate, decyl methacrylate, isodecyl methacrylate and dodecyl methacrylate, and mixtures thereof.

Additionally, crosslinked polymers may be chosen according to further exemplary embodiments. For example, such polymers may be chosen from polymers resulting from the polymerization of a mixture of monomers comprising:
(1) acrylic acid,
(2) an ester of formula (II) described above, in which R₂ is chosen from H or CH₃, R₃ denoting an alkyl radical having from 12 to 22 carbon atoms, and
(3) a crosslinking agent, which is a well-known copolymerizable polyethylenic unsaturated monomer, such as diallyl phthalate, allyl (meth)acrylate, divinylbenzene, (poly)ethylene glycol dimethacrylate and methylenebisacrylamide.

For example, acrylate/C₁₀-C₃₀ alkyl acrylate copolymers (INCI name: Acrylates/C₁₀₋₃₀ Alkyl Acrylate Crosspolymer), such as the products sold by Lubrizol under the trade names PEMULEN TR1, PEMULEN TR2, CARBOPOL 1382 and CARBOPOL EDT 2020 may be chosen.

Anionic polymers useful herein include, for example: Polyacrylic acid; Polymethacrylic acid; Carboxyvinylpolymer; acrylate copolymers such as Acrylate/C 10-30 alkyl acrylate crosspolymer, Acrylic acid/vinyl ester copolymer/AcrylatesNinyl Isodecanoate crosspolymer, Acrylates/Palmeth-25 Acrylate copolymer, Acrylate/Steareth-20 Itaconate copolymer, and Acrylate/Celeth-20 Itaconate copolymer; sulfonate polymers such as Polysulfonic acid, Sodium Polystyrene Sulfonate supplied from Akzo Nobel under the tradename FLEXAN II, copolymers of methacrylic acid and acrylamidomethylpropane sulfonic acid, and copolymers of acrylic acid and acrylamidomethylpropane sulfonic acid; carboxymethycellulose; carboxy guar gum; copolymers of ethylene and maleic acid; and acrylate silicone polymer. In some instances, the anionic polymers include, for example, Carbomer supplied from Noveon under the tradename CARBOPOL 981 and CARBOPOL 980; Acrylates/C10-30 Alkyl Acrylate Crosspolymer having tradenames Pemulen TR-1, PEMULEN TR-2, CARBOPOL 1342, CARBOPOL 1382, and CARBOPOL ETD 2020, all available from Noveon; sodium carboxymethylcellulose supplied from Hercules as CMC series; and Acrylate copolymer having a tradename Capigel supplied from Seppic; acrylates copolymer having the tradename CARBOPOL Aqua SF-1 and available from Lubrizol as an aqueous dispersion, and acrylates crosspolymer-4 having the tradename CARBOPOL Aqua SF-2 and available from Lubrizol as an aqueous dispersion.

In an embodiment, the anionic polymer of the invention is carbomer which may be commercially available from the supplier Lubrizol under the tradename of CARBOPOL 980.Exemplary of non-ionic polymers could be as follows:
(i) hydroxyethylcellulose, for instance the product NATROSOL 250 HHR PC or NATROSOL 250 HHR CS sold by the company Ashland;
(ii) celluloses modified with groups comprising at least one fatty chain; examples that may be mentioned include:
   hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product NATROSOL Plus Grade 330 CS (016 alkyls) sold by the company Ashland, or the product BERMOCOLL EHM 100 sold by the company AkzoNobel; methyl hydroxyethylcellulose; methyl ethyl hydroxyethylcellulose, known as the product STRUCTURE CEL 8000 M sold by the company AkzoNobel; or hydroxypropyl cellulose, known as the product KLUCEL MF PHARM HYDROXYPROPYLCELLULOSE sold by the company Ashland;
   hydroxyethylcelluloses modified with alkylphenyl polyalkylene glycol ether groups, such as the product Amercell Polymer HM-1500 (nonylphenyl polyethylene glycol (15) ether) sold by the company Amerchol; or
(iii) hydroxypropyl guars such as hydroxypropyl guar sold by as the product JAGUAR HP 105 by the company Rhodia and hydroxypropyl guars modified with groups comprising at least one fatty chain, such as the product Esaflor HM 22 (C₂₂ alkyl chain) sold by the company Lamberti, and the products RE210-18 (C₁₄ alkyl chain) and RE205-1 (C₂₀ alkyl chain) sold by the company Rhodia.

Emulsifying Polymer of the Polyoxyalkylenated Glycol Fatty Acid Ester Type

A further compound of the compositions according to the invention is an emulsifying polymer of the polyoxyalkylenated glycol fatty acid ester type.

The fatty acid ester of the said polymer is preferably polyhydroxylated. In particular, this polymer is a block polymer, preferably of structure ABA, comprising poly(hydroxylated ester) blocks and polyethylene glycol blocks.

The fatty acid ester of the said emulsifying polymer as defined above generally bears a chain comprising from 12 to 20 carbon atoms and preferably from 14 to 18 carbon atoms. The esters may be chosen especially from the oleates, palmitates and stearates.

The polyethylene glycol blocks of the said emulsifying polymer as defined above preferably comprise from 4 to 50 mol of ethylene oxide and more preferably from 20 to 40 mol of ethylene oxide.

A compound that is particularly suitable for producing the compositions of the invention is the 30 OE polyethylene glycol di-polyhydroxystearate sold under the trade name Arlacel P 135 by the company ICI.

The amount of polymers in the compositions according to the invention in a content ranging from 0.5% to 10% by weight relative to the total weight of the composition and more preferentially from 1.5% to 8% by weight relative to the total weight of the composition.

Polyamide

The polyamide used in the present invention includes aliphatic polyamides, for example polyamide-4, polyamide-6, polyamide-8, polyamide-11, polyamide-12, polyamide-4,6, polyamide-6,6, polyamide-6,9, polyamide-6,10, and polyamide-6,12; polyamides derived from an aliphatic diamine and an aromatic dicarboxylic acid, for example polyamide-4,T, polyamide-6,T, polyamide-4,l, etc., in which T stands for terephthalate and I stands for isophthalate; copolyamides of linear polyamides and copolyamides of an aliphatic and a partially aromatic polyamide, for example 6/6, T, 6/6,6/6, T, as well as amorphous polyamides of the Trogamid® PA 6-3-T and Grilamid® TR 55 types.

Preferably, the polyamides are selected from aliphatic polyamides, such as polyamide-4, polyamide-6, polyamide-8, polyamide-11, polyamide-12, polyamide-4,6, polyamide-6,6, polyamide-6, 9, and polyamide-6,10, polyamide-6,12.

Preferably, the polyamides may be aliphatic polyamides contain dimer acid(s). The dimer acid included in the aliphatic polyamides is preferably a dimer of fatty acids, preferably linear or branched, saturated or unsaturated C₆-C₃₀ fatty acids, which are optionally substituted with one or more hydroxyl groups. More preferably, the dimer acid is a dimer of unsubstituted, linear, and saturated C₆-C₃₀ fatty acids, such as hydrogenated linoleic acids.

In a preferable embodiment of the present invention, the polyamide is aliphatic polyamides terminated with (a) mono valent acid(s) and/or (a) mono valent alcohol(s). The mono valent acid may be a monovalent fatty acid, preferably linear or branched, saturated or unsaturated C₆-C₃₀ fatty acids, which are optionally substituted with one or more hydroxyl groups. The mono valent alcohol may be a monovalent fatty alcohol, preferably nonoxyalkylenated, saturated or unsaturated, linear or branched, C₆ to C₃₀ fatty alcohol. More preferably, the mono valent alcohol may be nonoxyalkylenated, saturated, and linear C₆ to C₃₀ fatty alcohol, such as stearyl alcohol.

Preferentially, the polyamide is aliphatic polyamides terminated with mono valent alcohols.

In particular, the polyamide is polyamide-8, under the chemical name of Bis-stearyl ethylenediamine/neopentyl glycol/stearyl hydrogenated dimer dilinoleate copolymer, wherein the polyamide-8 is such as Oleocraft LP-20-PA-(MV) sold by Croda.

In a preferred embodiment, the polyamide-8 is a gelling agent/oil-structuring agent/film-former.

The amount of polyamide in the composition according to the present invention may be from 1 to 10% by weight, preferably from 1.5 to 8% by weight, more preferably from 1.5 to 6% by weight relative to the total weight of the composition.

Fillers

Suitable fillers of the invention could be as examples of oil-absorbing fillers: mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch.

The amount of fillers in the composition according to the present invention may be from 1 to 15% by weight, preferably from 1.5 to 10% by weight, more preferably from 1.5 to 8% by weight relative to the total weight of the composition.

Silica Silylate

The “silica silylate” according to the present invention is a porous material obtained by replacing (by drying) the liquid component of a silica gel with air. Silica aerogels are generally synthesized via a sol-gel process in a liquid medium and then dried, usually by extraction with a supercritical fluid, such as, but not limited to, supercritical carbon dioxide (CO₂). This type of drying makes it possible to avoid shrinkage of the pores and of the material. The sol-gel process and the various drying processes are described in detail in Brinker, C. J., and Scherer, G. W., Sol-Gel Science: New York: Academic Press, 1990.

The silica silylate particles used in the present invention have a specific surface area per unit of mass (S_{M}) ranging from about 500 to about 1500 m²/g, or alternatively from about 600 to about 1200 m²/g, or alternatively from about 600 to about 800 m²/g, and a size expressed as the mean volume diameter (D[0.5]), ranging from about 1 to about 30 μm, or alternatively from about 5 to about 25 μm, or alternatively from about 5 to about 20 μm, or alternatively from about 5 to about 15 μm. The specific surface area per unit of mass may be determined via the BET (Brunauer-Emmett-Teller) nitrogen absorption method described in the Journal of the American Chemical Society, vol. 60, page 309, February 1938, corresponding to the international standard ISO 5794/1. The BET specific surface area corresponds to the total specific surface area of the particles under consideration.

The size of the silica silylate particles may be measured by static light scattering using a commercial granulometer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles,” Chapters 9 and 10, Wiley, New York, 1957.

The silica silylate particles used in the present invention may advantageously have a tamped (or tapped) density ranging from about 0.04 g/cm³ to about 0.10 g/cm³, or alternatively from about 0.05 g/cm³ to about 0.08 g/cm³. In the context of the present invention, this density, known as the tamped density, may be assessed according to the following protocol: 40 g of powder are poured into a measuring cylinder; the measuring cylinder is then placed on a Stay 2003 machine from Stampf Volumeter; the measuring cylinder is then subjected to a series of 2500 packing motions (this operation is repeated until the difference in volume between two consecutive tests is less than 2%); the final volume Vf of packed powder is then measured directly on the measuring cylinder. The tamped density is determined by the ratio m/Vf, in this instance 40/Vf (Vf being expressed in cm³ and m in g).

According to one embodiment, the silica silylate particles used in the present invention have a specific surface area per unit of volume Sv ranging from about 5 to about 60 m²/cm³, or alternatively from about 10 to about 50 m²/cm³, or alternatively from about 15 to about 40 m²/cm³. The specific surface area per unit of volume is given by the relationship: Sv=S_{M.}r where r is the tamped density expressed in g/cm³ and S_{M} is the specific surface area per unit of mass expressed in m²/g, as defined above.

In some embodiments, the silica silylate particles, according to the invention, have an oil-absorbing capacity, measured at the wet point, ranging from about 5 to about 18 ml/g, or alternatively from about 6 to about 15 ml/g, or alternatively from about 8 to about 12 ml/g. The oil-absorbing capacity measured at the wet point, noted Wp, corresponds to the amount of water that needs to be added to 100 g of particle in order to obtain a homogeneous paste. Wp is measured according to the wet point method or the method for determining the oil uptake of a powder described in standard NF T 30-022. Wp corresponds to the amount of oil adsorbed onto the available surface of the powder and/or absorbed by the powder by measuring the wet point, described below: An amount=2 g of powder is placed on a glass plate, and the oil (isononyl isononanoate) is then added dropwise. After addition of 4 to 5 drops of oil to the powder, mixing is performed using a spatula, and addition of oil is continued until a conglomerate of oil and powder has formed. At this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in ml) of oil used is then noted. The oil uptake corresponds to the ratio Vs/m.

The silica silylate according to the present invention, is a hydrophobic silica aerogel. The term “hydrophobic silica” means any silica whose surface is treated with silylating agents, for example, halogenated silanes, such as alkylchlorosilanes, siloxanes, in particular dimethylsiloxanes, such as hexamethyldisiloxane, or silazanes, so as to functionalize the OH groups with silyl groups Si—Rn, for example, trimethylsilyl groups. Preparation of hydrophobic silica aerogel particles that have been surface-modified by silylation, is found in U.S. Pat. No. 7,470,725, incorporated herein by reference. In one embodiment, silica silylate particles surface-modified with trimethylsilyl groups are desirable.

Suitable examples of silica silylate, may include, but are not limited to, the aerogels sold under the tradenames of VM-2260 (INCI name: Silica silylate) and VM-2270 (INCI name: Silica silylate), both available from Dow Corning Corporation (Midland, Michigan). The particles of VM-2260 have a mean size of about 1000 microns and a specific surface area per unit of mass ranging from 600 to 800 m²/g. The particles of VM-2270 have a mean size ranging from 5 to 15 microns and a specific surface area per unit of mass ranging from 600 to 800 m²/g. Another suitable example of a hydrophobic silica aerogel may include, but is not limited to, the aerogels commercially available from Cabot Corporation (Billerica, Massachusetts) under the tradename of Aerogel TLD 201, Aerogel OGD 201 and Aerogel TLD 203, Enova Aerogel MT 1100 and Enova Aerogel MT 1200.

The silica silylate is preferably hydrophobic silica aerogel.

Fatty Compounds

The cosmetic sunscreen compositions may include fatty compounds. A “fatty compound” is generally an organic compound that is not soluble in water at normal temperature (25° C.) and at atmospheric pressure (750 mmHg) (solubility below 10%). In some instances, the solubility in water may be below 5%, below 1%, or below 0.1%). Moreover, fatty compounds are generally soluble in one or more organic solvents under the same conditions of temperature and pressure, for example organic solvents such as chloroform, ethanol, benzene or decamethylcyclopentasiloxane.

Non-limiting examples of fatty compounds include oils, mineral oil, fatty alcohol derivatives, fatty acid derivatives (such as polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, waxes, triglyceride compounds, lanolin, and a mixture thereof. In some instances, the at least one fatty compound includes one or more esters of fatty acids, and/or esters of fatty alcohols (for example, cetyl palmitate, cetyl stearate, myristyl myristate, myristyl stearate, cetyl myristate, and stearyl stearate (a mixture of which is referred to as “cetyl esters”)). The fatty compounds may be liquid or solid at room temperature and at atmospheric pressure (25° C., 1 atm).

In some cases, the one or more fatty compounds may be one or more high melting point fatty compounds. A high melting point fatty compound is a fatty compound having a melting point of greater than 25° C. Even higher melting point fatty compounds may also be used, for example, fatty compounds having a melting point of 30° C. or higher, 40° C. or higher, 45° C. or higher, 50° C. or higher. Non-limiting examples of the high melting point compounds are found in International Cosmetic Ingredient Dictionary, Fifteenth Edition, 2014, which is incorporated herein by reference in its entirety.

Additional and/or alternative fatty alcohol derivatives include alkyl ethers of fatty alcohols, alkoxylated fatty alcohols, alkyl ethers of alkoxylated fatty alcohols, esters of fatty alcohols and a mixture thereof. Nonlimiting examples of fatty alcohol derivatives include materials such as methyl stearyl ether; 2-ethylhexyl dodecyl ether; steelyl acetate; cetyl propionate; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and steelyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Non-limiting examples of hydrocarbons include linear or branched, optionally cyclic C₆-C₁₆ alkanes; hexane, undecane, dodecane, tridecane, and isoparaffins, for instance isohexadecane, isododecane and isodecane. Additionally, the linear or branched hydrocarbons may be composed only of carbon and hydrogen atoms of mineral, plant, animal or synthetic origin with more than 16 carbon atoms, such as volatile or non-volatile liquid paraffins, petroleum jelly, liquid petroleum jelly, polydecenes, hydrogenated polyisobutene, and squalane.

The amount of fatty compounds present in the sunscreen cosmetic composition may range from, e.g., ranging from about 1% to 12% by weight and preferably from about 1.5% to about 10% by weight, more preferably from about 1.5% to about 8% by weight, including all ranges and sub-ranges there between, based on the total weight of the cosmetic composition.

Fatty Ester(s)

Non-limiting examples of liquid fatty esters include esters from a C₆-C₃₂ fatty acid and/or a C₆-C₃₂ fatty alcohol, and are liquid at 25° C., 1 atm. These esters may be liquid esters of saturated or unsaturated, linear or branched C₁-C₂₆ aliphatic mono or polyacids and of saturated or unsaturated, linear or branched C₁-C₂₅ aliphatic mono or polyalcohols, the total number of carbon atoms in the esters being greater than or equal to 10. In some cases, for the esters of monoalcohols, at least one of the alcohol or the acid from which the esters of the invention result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate and isostearyl neopentanoate.

In some cases, it is particularly useful to include cetyl esters in the hair conditioning compositions. Cetyl Esters is a mixture of the following esters of saturated fatty acids and fatty alcohols: cetyl palmitate, cetyl stearate, myristyl myristate, myristyl stearate, cetyl myristate, and stearyl stearate.

Esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate; diisopropyl sebacate; diisopropyl adipate; di-n-propyl adipate; triisopropyl citrate; glyceryl trilactate; glyceryl trioctanoate; neopentyl glycol diheptanoate; and diethylene glycol diisononanoate.

Non-limiting liquid fatty esters that may be mentioned include, for example, sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, coconut oil, camellina oil, tamanu oil, babassu oil and pracaxi oil, jojoba oil, and shea butter oil.

The solid fatty acid esters and/or fatty acid esters that may be mentioned include solid esters obtained from C₉-C₂₆ fatty acids and from C₉-C₂₅ fatty alcohols. Among these esters, mention may be made of octyldodecyl behenate, isocetyl behenate, cetyl lactate, steelyl octanoate, octyl octanoate, cetyl octanoate, decyl oleate, myristyl stearate, octyl palmitate, octyl pelargonate, octyl stearate, alkyl myristates such as cetyl myristate, myristyl myristate or steelyl myristate, and hexyl stearate.

Fatty Ether(s)

The liquid fatty ethers may be chosen from liquid dialkyl ethers such as dicaprylyl ether. The non-liquid fatty ethers may also be chosen from dialkyl ethers and in particular dicetyl ether and distearyl ether, alone or as a mixture.

Non-limiting examples of waxes include carnauba wax, candelilla wax, esparto wax, paraffin wax, ozokerite, plant waxes such as olive tree wax, rice wax, hydrogenated jojoba wax or absolute flower waxes, such as the blackcurrant blossom essential wax sold by Bertin (France), or animal waxes such as beeswaxes or modified beeswaxes (cerabellina), and ceramides. Non-limiting examples of ceramides include N-linoleyldihydrosphingosine, N-oleyldihydrosphingosine, N-palmityldihydrosphingosine, N-stearyldihydrosphingosine or N-behenyldihydrosphingosine, or mixtures of these compounds.

Fatty Acid Derivatives

The fatty acid derivatives may include, but are not limited to, ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof.

Ester Oil(s)

The one or more ester oils may be or include one or more monoester oils, one or more diester oils, one or more triester oils, or a combination thereof. In some instances, the one or more esters oils include one or more monoester oils, one or more diester oils, or a combination thereof. In some instances, the one or more ester oils include one or more diesters. For purposes of the present application triglycerides are not considered ester oils.

Non-limiting examples of the one or more diester oils include those chosen from diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, diisostearyl fumarate, and mixtures thereof.

Additional non-limiting examples of ester oils include, for example, as monoesters, isononanoates such as isononyl isononanoate and isotridecyl isononanoate, etc., 2-ethylhexanoate such as cetyl ethylhexanoate and hexyldecyl ethylhexanoate, etc., myristates such as isopropyl myristate, isocetyl myristate, octyldodecyl myristate, etc., isostearates such as ethyl isostearate, isopyropyl isostearate, hexyldecyl isostearate, isostearyl isostearate, cholesteryl isostearate, phytosteryl isostearate, etc., lactates such as isostearyl lactate, octyldodecyl lactate, etc., hydroxystearates such as ethylhexyl hydroxystearate, octyl hydroxystearate, phytosteryl hydroxystearate, cholesteryl hydroxystearate, etc., oleates such as oleyl oleate, phytosteryl oleate, octyldodecyl oleate, etc., neopentanoates such as isodecyl neopentanoate, isostearyl neopentanoate, etc., palmitates such as isopyropyl palmitate, ethylhexyl palmitate, etc., and octyldodecyl neodecanoate, octyldodecyl ricinoleate, oleyl erucate, octyldodecyl erucate, isopropyl lauroylsarcosinate, etc.

Non-limiting examples of diester oils include diisobutyl adipate, diisopropyl adipate, diethylhexyl succinate, neopentyl glycol diisononanoate, neopentyl glycol diethyl hexanoate, neopentyl glycol dicaprate, diisostearyl malate, diisopropyl dilinoleate, ethylene glycol dioctanoate, octyldodecyl stearoyloxystearate, diisopropyl sebacate, di(cholesteryl/octyldodecyl) lauroyl glutamate, di(phytosteryl/octyldodecyl) lauroyl glutamate, etc.

The one or more ester oils may be or include one or more triester oils. Examples of triester oils that may, optionally be used, include triethyl hexanoin, trimethylolpropane triethylhexanoate, triisostearin, trimethylolpropane triisostearate, etc. Tetraester oils include pentaerythrityl tetraethylhexanoate, pentaerythrityl tetraisostearate, etc.

The one or more ester oils may be or include one or more polyester oils. Non-limiting examples of polyester oils include polyglycerin fatty acid esters such as polyglyceryl-2 isostearate, polyglyceryl-2 diisostearate, polyglyceryl-2 triisostearate, polyglyceryl-2 tetraisostearate, etc.

The ester oils may also be high-viscosity ester oils such as those chosen from dipentaerythrityl hexa(hydroxystearate/stearate/rosinate), hydrogenated castor oil isostearate, hydrogenated castor oil dimer dilinoleate, (polyglyceryl-2 isostearate/dimer dilinoleate) copolymer, (phytosteryl/isostearyl/cetyl/stearyl/behenyl) dimer dilinoleate, bis(phytosteryl/behenyl/isostearyl) dimer dilinoleyl dimer dilinoleate, di(isostearyl/phytosteryl) dimer dilinoleate, dimer dilinoleyl hydrogenated rosin condensation product, dimer dilinoleyl diisostearate, dimer dilinoleyl dimer dilinoleate, di(cholesteryl/behenyl/octyldodecyl) lauroyl glutamate, di(octyldodecyl/phytosteryl/behenyl) lauroyl glutamate, myristoyl methylalanine (phytosteryl/decyl tetradecyl), (diglycerin/dilinoleate/hydroxystearate) copolymer, etc.

Emulsified Carnauba Wax

As used herein, the “emulsified carnauba wax” relates to an oil-in-water-emulsion (O/W), i.e., an emulsified carnauba wax, that comprises solid carnauba wax, water and at least one surfactant, marketed under the trademark Hostapur CW by Clariant.

In a preferred embodiment, the amount of solid carnauba wax in the O/W emulsion is at least about 30% by weight, relative to the total weight of the 0/W emulsion, preferably from about 30% by weight to about 50% by weight, based on the total weight of the O/W emulsion.

Solid carnauba wax is a hard wax scraped from the leaves and leaf stems of carnauba palms, Copernicia cerifera. The carnauba wax comprises esters of C₁₈-C₃₂ fatty acids, and C₂₈-C₃₄ alcohols, also containing high amounts of hydroxy acid esters and melting points around 80 and 86° C.

Additionally, the solid carnauba wax usually comprises from about 80% by weight to about 85% by weight of fatty esters, from about 1% by weight to about 5% by weight of alcohols, from about 1% by weight to about 5% by weight of hydrocarbons, from about 1% by weight to about 5% by weight of free acids, from about 1% by weight to about 6% by weight of resins, from about 1% by weight to about 5% by weight of lactic components and from about 0.1% by weight to about 2% by weight of humidity.

In a preferred embodiment, the amount of water in the O/W emulsion of the present invention is at least about 40% by weight, relative to the total weight of the O/W emulsion, preferably from about 40% by weight to about 60% by weight, based on the total weight of the O/W emulsion.

The at least one surfactant of the O/W emulsion is selected from the group of anionic surfactants, non-ionic surfactants and mixtures thereof.

Non-limiting examples of anionic surfactants for the embodiment of the present invention are selected from the group comprising alkyl sulfates, alkyl phosphates, alkyl ether sulfates, alkyl ether phosphates, alkylamido ether sulfates, alkylaryl polyether sulfates, monoglyceride sulfates, sulfonates, such as alkylsulfonates, alkylamide sulfonates, alkylarylsulfonates, alpha-olefin sulfonates, paraffin sulfonates, sulfosuccinates, alkylsulfosuccinates, alkyl ether sulfosuccinates, alkylamide sulfosuccinates, alkyl sulfoacetates, acylsarcosinates, acylglutamates, alkylsulfosuccinamates, taurates and N-acyl N-methyltaurates, isethionates, N-acylisethionates, N-acyltaurates, phosphates and alkyl phosphates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

Non limiting examples of non-ionic surfactants for the embodiment of the present invention include, for example, alkyl- and polyalkyl-esters of glycerol, such as polyglyceryl-3 dicitrate/stearate, mixtures of alkyl- and polyalkyl-esters of glycerol with polyglyceryl, such as polyglyceryl-3 methylglucose distearate, oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters, for instance sucrose stearate; fatty alcohol ethers of sugars, especially alkyl polyglucosides (APGs) such as decyl glucoside, lauryl glucoside, cetostearyl glucoside, optionally as a mixture with cetostearyl alcohol, and also arachidyl glucoside, for example in the form of a mixture of arachidyl alcohol, behenyl alcohol and arachidyl glucoside. According to one particular embodiment of the invention, the mixture of the alkyl polyglucoside as defined above with the corresponding fatty alcohol may be in the form of a self-emulsifying composition. Mention may also be made of lecithins and derivatives (e.g. Biophilic), sugar esters and sodium stearoyl lactylate.

In a preferred embodiment, the surfactants used in the O/W emulsion are isotridecyl phosphate and laureth-23.

The emulsified carnauba wax of the present invention may also comprise additional ingredients such as preserving agents and solvents. Non-limiting example of preserving agent which can be used in accordance with the invention includes phenoxyethanol. Non-limiting example of solvent which can be used in accordance with the invention includes glycerin.

The amount of emulsified carnauba wax in the anhydrous cosmetic sunscreen composition of the present invention is preferably at least about 1% by weight, relative to the total weight of the composition, more preferably from about 1% by weight to about 10% by weight, even more preferably from about 1% by weight to about 8% by weight, based on the total weight of the composition.

Surfactant

Anionic Surfactants

Anionic surfactants mean a surfactant comprising, as ionic or ionizable groups, only anionic groups. These anionic groups are chosen preferably from the groups CO₂H, CO₂—, SO₃H, SO₃—, OSO₃H, OSO₃—O₂PO₂H, O₂PO₂H and O₂PO₂²—.

Non-limiting anionic surfactant(s) that may be used in the present invention are selected from the group comprising alkyl sulfates, alkyl ether sulfates, alkylamido ether sulfates, alkylaryl polyether sulfates, monoglyceride sulfates, sulfonates, such as alkylsulfonates, alkylamide sulfonates, alkylarylsulfonates, alpha-olefin sulfonates, paraffin sulfonates, sulfosuccinates, alkylsulfosuccinates, alkyl ether sulfosuccinates, alkylamide sulfosuccinates, alkyl sulfoacetates, acylsarcosinates, acylglutamates, alkylsulfosuccinamates, taurates and N-acyl N-methyltaurates, isethionates, N-acylisethionates, N-acyltaurates, phosphates and alkyl phosphates, salts of alkyl monoesters and polyglycoside-polycarboxylic acids, acyllactylates, mixed esters of organic acids with glycerol, such as glyceryl stearate citrate and as glyceryl stearate lactate, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

Further examples of anionic surfactants also comprise the dimeric surfactant of formula (I), described in WO 96/14926:

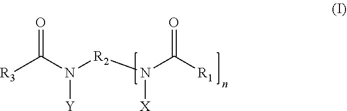

in which:
R₁ and R₃ denote, independently of each other, an alkyl radical containing from 1 to 25 carbon atoms;
R₂ denotes a spacer consisting of a linear or branched alkylene chain containing from 1 to 12 carbon atoms;
X denotes a group —(C₂H₄O)ₐ—(C₃H₆O)_{b}Z,
Y denotes a group —(C₂H₄O)_{c}—(C₃H₆O)_{d}Z,
in which
Z denotes a hydrogen atom or a radical —CH₂—COOM, —SO₃M, —P(O)(OM)₂, —C₂H₄SO₃M, or —C₃HsSO₃M group, in which M & M′ represent H or an alkali metal or alkaline-earth metal or ammonium or alkanolammonium ion,
a and c, independently of each other, range from 0 to 15,
b and d, independently of each other, range from 0 to 10, and
the sum of a+b+c+d ranges from 1 to 25; and n ranges from 1 to 10.

A surfactant of this type is, particularly, the one identified by the INCI name: Disodium ethylene dicocamide PEG-15 disulfate, having the following structure:

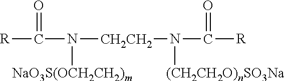

wherein RCO represents a coconut fatty acid radical and m+n has a mean value of 15.

In a preferred embodiment, the anionic surfactants of the present invention are select from disodium ethylene dicocamide PEG-15 disulfate, glyceryl stearate citrate and mixtures thereof.

Non-Ionic Surfactants

Non-limiting examples of non-ionic surfactants useful in the present invention include, for example, alkyl- and polyalkyl-esters of glycerol, such as polyglyceryl-3 dicitrate/stearate, mixtures of alkyl- and polyalkyl-esters of glycerol with polyglyceryl, such as polyglyceryl-3 methylglucose distearate, oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters, for instance sucrose stearate; fatty alcohols, fatty alcohol ethers of sugars, especially alkyl polyglucosides (APGs) such as decyl glucoside, lauryl glucoside, cetostearyl glucoside, optionally as a mixture with cetostearyl alcohol, and also arachidyl glucoside, for example in the form of a mixture of arachidyl alcohol, behenyl alcohol and arachidyl glucoside. According to one particular embodiment of the invention, the mixture of the alkyl polyglucoside as defined above with the corresponding fatty alcohol may be in the form of a self-emulsifying composition. Mention may also be made of lecithins and derivatives (e.g. Biophilic), sugar esters and sodium stearoyl lactylate.

In a preferred embodiment, the nonionic surfactants may be chosen from:
C₈-C₃₀ fatty alcohol ethers of a sugar, in particular (C₈-C₃₀)alkyl (poly)glucosides,
ethers of polyethylene glycol, in particular containing from 15 to 25 ethylene oxide units, and of a C₈-C₃₀ fatty acid ester of glucose or of methylglucose,
C₈-C₃₀ fatty acid esters of sorbitan,
polyoxyethylenated C₈-C₃₀ fatty acid esters of sorbitan, especially containing from 2 to 20 mol of ethylene oxide,
polyoxyethylenated C₈-C₃₀ fatty acid esters of sorbitan, especially containing from 2 to 20 mol of ethylene oxide,
C₈-C₃₀ fatty acid monoesters or diesters of glycerol,
polyglycerolated C₈-C₃₀ fatty acid esters, especially containing from 2 to 16 mol of glyc-erol,
C₈-C₃₀ fatty acid esters of polyethylene glycol, especially containing from 2 to 20 eth-ylene oxide units,
C₈-C₃₀ fatty acid esters of glucose or of (C₁-C₂)alkylglucose or of sucrose,
and mixtures thereof;

Preferably, the nonionic surfactant can be chosen from (C₈-C₃₀)alkyl (poly)glucosides.

The nonionic surfactants may also be chosen from C₈-C₃₀ fatty alcohol ethers of a sugar, in particular (C₈-C₃₀)alkyl (poly)glucosides.

The alkyl (poly)glucoside may be chosen from a group comprising the compounds having the following general formula:

R₁O—(G)ₐ
wherein R1 denotes a linear or branched alkyl radical comprising from 8 to 30 carbon at-oms and preferably from 8 to 24 carbon atoms, the G group denotes a sugar comprising from 5 to 6 carbon atoms and a is a number ranging from 1 to 10, and mixtures thereof.

The alkyl (poly)glucoside may be chosen especially from the group comprising C₈-C₂₂ fat-ty alcohol ethers or mixtures of ethers of glucose or of xylose, preferably of glucose.

The unit (or chain) derived from the fatty alcohol of the ethers may be chosen especially from caprylyl, capryl, decyl, lauryl, myristyl, cetyl (or palmityl), stearyl, octyldodecyl, ara-chidyl, behenyl and hexadecanoyl units, and mixtures thereof such as cetearyl.

In a particular embodiment, the alkyl (poly)glucoside is chosen from caprylyl/capryl gluco-side, decyl glucoside, lauryl glucoside, myristyl glucoside, cetearyl glucoside, arachidyl glucoside, cocoyl glucoside, octyldodecyl glucoside, caprylyl/capryl xyloside, octyldodecyl xyloside, and a mixture thereof, preferably cetearyl glucoside and arachidyl glucoside.

Examples of alkyl (poly)glucosides that may be mentioned include caprylyl/capryl gluco-side, for instance the product sold under the name Oramix CG 110 by the company SEPPIC, decyl glucoside sold, for example, under the names Plantaren 2000 by the com-pany Henkel, Plantacare 2000 UP by the company Cognis, Mydol 10 by the company Kao, or Oramix NS 10 by the company SEPPIC, lauryl glucoside sold, for example, by the company Henkel under the name Plantaren 1200, coco glucoside sold, for example, under the name Plantacare 818 UP by the company Cognis, cetearyl glucoside optionally as a mixture with cetearyl alcohol, sold, for example, under the name Montanov 68 by the company SEPPIC or under the name Xyliance by the company Soliance, under the name Tego Care CG90 by the company Evonik Goldschmidt and under the name Emulgade KE 3302 by the company Henkel, and also arachidyl glucoside, for example in the form of the mixture of arachidyl and behenyl alcohols and of arachidyl glucoside, sold under the name Montanov 202 by the company SEPPIC, the mixture of cocoyl polyglucoside and of cetyl and stearyl alcohols (35/65) sold, for example, under the name Montanov 82 by the com-pany SEPPIC, octyldodecyl xyloside sold under the names Fluidanov 20X or Easynov by the company SEPPIC, myristyl glucoside, and especially in the form of a mixture with myristyl alcohol, for instance the product sold by the company SEPPIC under the name Montanov 14, mixtures of (C12-C20)alkyl glucosides especially as a mixture with C14 to C22 fatty alcohols, for instance the mixture sold under the name Montanov L by the com-pany SEPPIC, and mixtures thereof.

For the W/O emulsions (water-in-oil or inverted emulsions), hydrocarbon-based or silicone surfactants may be used.

According to one embodiment variant, hydrocarbon-based surfactants are preferred.

Examples of hydrocarbon-based surfactants that may be mentioned include polyester polyols, for instance PEG-30 dipolyhydroxystearate sold under the reference Arlacel P 135 by the company Uniqema, and polyglyceryl-2 dipolyhydroxystearate sold under the reference Dehymuls PGPH by the company Cognis.

Examples of silicone surfactants that may be mentioned include alkyl dimethicone copolyols such as lauryl methicone copolyol sold under the name Dow Corning 5200 Formulation Aid by the company Dow Corning and cetyl dimethicone copolyol sold under the name Abil EM 90 by the company Goldschmidt, or the polyglyceryl-4 isostearate/cetyl dimethicone copolyol/hexyl laurate mixture sold under the name Abil WE 09 by the company Goldschmidt.

One or more co-emulsifiers may also be added thereto. The co-emulsifier may be chosen advantageously from the group comprising polyol alkyl esters. Polyol alkyl esters that may especially be mentioned include glycerol and/or sorbitan esters, for example the polyglyceryl-3 diisostearate sold under the name Lameform TGI by the company Cognis, polyglyceryl-4 isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

The amount of surfactants in the composition according to the present invention may be from 1 to 10% by weight, preferably from 1.5 to 10% by weight, more preferably from 1.5 to 8% by weight, and in particular from 1.5 to 6% by weight relative to the total weight of the composition.

UV Filters

Inorganic UV Filters

The composition, according to the present invention, comprise a UV filter system comprising at least one inorganic UV filter. If two or more inorganic UV filters are used, they may be the same or different.

The inorganic UV filter used for the present invention may be active in the UV-A and/or UV-B region. The inorganic UV filter may be hydrophilic and/or lipophilic. The inorganic UV filter is in some embodiments insoluble in solvents, such as water, and ethanol commonly used in cosmetics.

It is in some embodiments desirable that the inorganic UV filter be in the form of a fine particle such that the mean (primary) particle diameter thereof ranges from about 1 nm to about 50 nm, and in some embodiments from about 5 nm to about 40 nm, and in some embodiments from about 10 nm to about 30 nm. The mean (primary) particle size or mean (primary) particle diameter here is an arithmetic mean diameter.

The inorganic UV filter can be selected from the group consisting of silicon carbide, metal oxides which may or may not be coated, and mixtures thereof. And in some embodiments, the inorganic UV filters are selected from pigments (mean size of the primary particles: generally from about 5 nm to about 50 nm, and in some embodiments from about 10 nm to about 50 nm) formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se. And in some embodiments, the inorganic UV filters are selected from titanium oxide, zinc oxide, and, in some embodiments, titanium oxide.

The inorganic UV filter may or may not be coated. The inorganic UV filter may have at least one coating. The coating may comprise at least one compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per)fluoro compounds. It is in some embodiments desirable for the coating to include at least one organic UV filter. As the organic UV filter in the coating, a dibenzoylmethane derivative, such as butyl methoxydibenzoylmethane (Avobenzone) and 2,2′-Methylenebis[6-(2H-Benzotriazol-2-yl)-4-(1,1,3,3-Tetramethyl-Butyl) Phenol] (Methylene Bis-Benzotriazolyl Tetramethylbutylphenol), such as marketed as “TINOSORB M” by BASF, may be desirable.

In a known manner, the silicones in the coating(s) may be organosilicon polymers or oligomers comprising a linear or cyclic and branched or cross-linked structure, of variable molecular weight, obtained by polymerization and/or polycondensation of suitable functional silanes and essentially composed of repeated main units in which the silicon atoms are connected to one another via oxygen atoms (siloxane bond), optionally substituted hydrocarbon radicals being connected directly to said silicon atoms via a carbon atom.

The term “silicones” also encompasses silanes necessary for their preparation, in particular alkylsilanes.

The silicones used for the coating(s) can be and in some embodiments are selected from the group consisting of alkylsilanes, polydialkylsiloxanes, and polyalkylhydrosiloxanes. And in some embodiments still, the silicones are selected from the group consisting of octyltrimethylsilane, polydimethylsiloxanes, and polymethylhydrosiloxanes.

Of course, the inorganic UV filters made of metal oxides may, before their treatment with silicones, have been treated with other surfacing agents, in particular with cerium oxide, alumina, silica, aluminum compounds, silicon compounds, or their mixtures. The coated inorganic UV filter may have been prepared by subjecting the inorganic UV filter to one or more surface treatments of a chemical, electronic, mechano-chemical, and/or mechanical nature with any of the compounds as described above, as well as polyethylene waxes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filters may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and in some embodiments TiO₂ treated with octyltrimethylsilane and for which the mean size of the individual particles is from 25 and 40 nm, such as that marketed under the trademark “T 805” by Degussa Silicas, TiO₂ treated with a polydimethylsiloxane and for which the mean size of the individual particles is 21 nm, such as that marketed under the trademark “70250 Cardre UF TiO₂Si₃” by Cardre, and anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO₂ can be used as the coated inorganic UV filter: Stearic acid (and) Aluminum Hydroxide (and) TiO₂, such as the product “MT-100 TV” from Tayca, with a mean primary particle diameter of 15 nm; Dimethicone (and) Stearic Acid (and) Aluminum Hydroxide (and) TiO₂, such as the product “S A-TTO-S4” from Miyoshi Kasei, with a mean primary particle diameter of 15 nm; Silica (and) TiO₂, such as the product “MT-100 WP” from Tayca, with a mean primary particle diameter of 15 nm; Dimethicone (and) Silica (and) Aluminum Hydroxide (and) TiO₂, such as the product “MT-Y02” and “MT-Y-110 M3S” from Tayca, with a mean primary particle diameter of 10 nm; Dimethicone (and) Aluminum Hydroxide (and) TiO₂, such as the product “SA-TTO-S3” from Miyoshi Kasei, with a mean primary particle diameter of 15 nm; Dimethicone (and) Alumina (and) TiO₂, such as the product “UV TITAN MI 70” from Sachtleben, with a mean primary particle diameter of 15 nm; and Silica (and) Aluminum Hydroxide (and) Alginic Acid (and) TiO₂, such as the product “MT-100 AQ” from Tayca, with a mean primary particle diameter of 15 nm. In terms of UV filtering ability, TiO₂ coated with at least one organic UV filter is more desirable. For example, Avobenzone (and) Stearic Acid (and) Aluminum Hydroxide (and) TiO₂, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxide pigments are, for example, those marketed under the trademark “Z-cote” by Sunsmart; those marketed under the trademark “Nanox” by Elementis; and those marketed under the trademark “Nanogard WCD 2025” by Nanophase Technologies. The coated zinc oxide pigments are, for example, those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C₁₂-C₁₅ alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with phosphate of perfiuoroalkyl and a copolymer based on perfluoroalkylethyl as a dispersion in cyclopentasiloxane); those marketed under the trademark “SPD-Z1” by Shin-Etsu (ZnO coated with a silicone-grafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C12-C15 alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”. Mention may also be made of mixtures of metal oxides, in particular, of titanium dioxide and of cerium dioxide, including a mixture of equal weights of titanium dioxide coated with silica and of cerium dioxide coated with silica, such as marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

Coated inorganic UV filters are desirable, because the UV filtering effects of the inorganic UV filters can be enhanced. In addition, the coating(s) may help uniformly or homogeneously disperse the UV filters in the composition, according to the present invention.

Organic UV Filters

The composition, according to the present invention, comprises a UV filter system comprising at least one organic UV filter. If two or more organic UV filters are used, they may be the same or different.

The organic UV filter used for the present invention may be active in the UV-A and/or UV-B region. The organic UV filter may be hydrophilic and/or lipophilic. The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; 6,6-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV filter(s), of those denoted below under their INCI names, and mixtures thereof. Anthranilic compounds: menthyl anthranilates, such as marketed under the trademark “Neo Heliopan MA” by Haarmann and Reimer. The dibenzoylmethane compounds: Butyl methoxydibenzoylmethane, such as marketed in particular under the trademark “Parsol 1789” by Hoffmann-La Roche; and isopropyl dibenzoylmethane. Cinnamic compounds: Ethylhexyl methoxycinnamate, such as marketed in particular under the trademark “Parsol MCX” by Hoffmann-La Roche; isopropyl methoxycinnamate; isopropoxy methoxycinnamate; isoamyl methoxycinnamate, such as marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), such as marketed under the trademark “Eusolex HMS” by Rona/EM Industries; ethylhexyl salicylate, such as marketed under the trademark “Neo Heliopan OS” by Haarmann and Reimer; glycol salicylate; butyloctyl salicylate; phenyl salicylate; dipropyleneglycol salicylate, such as marketed under the trademark “Dipsal” by Scher; and TEA salicylate, such as marketed under the trademark “Neo Heliopan TS” by Haarmann and Reimer. Camphor compounds, in particular, benzylidenecamphor derivatives: 3-benzylidene camphor, such as manufactured under the trademark “Mexoryl SD” by Chimex; 4-methylbenzylidene camphor, such as marketed under the trademark “Eusolex 6300” by Merck; benzylidene camphor sulfonic acid, such as manufactured under the trademark “Mexoryl SL” by Chimex; camphor benzalkonium methosulfate, such as manufactured under the trademark “Mexoryl SO” by Chimex; terephthalylidene dicamphor sulfonic acid, such as manufactured under the trademark “Mexoryl SX” by Chimex; and polyacrylamidomethyl benzylidene camphor, such as manufactured under the trademark “Mexoryl SW” by Chimex. Benzophenone compounds: Benzophenone-1 (2,4-dihydroxybenzophenone), such as marketed under the trademark “Uvinul 400” by BASF; benzophenone-2 (Tetrahydroxybenzophenone), such as marketed under the trademark “Uvinul D50” by BASF; Benzophenone-3 (2-hydroxy-4-methoxybenzophenone) or oxybenzone, such as marketed under the trademark “Uvinul M40” by BASF; benzophenone-4 (hydroxymethoxy benzophonene sulfonic acid), such as marketed under the trademark “Uvinul MS40” by BASF; benzophenone-5 (Sodium hydroxymethoxy benzophenone Sulfonate); benzophenone-6 (dihydroxy dimethoxy benzophenone); such as marketed under the trademark “Helisorb 11” by Norquay; benzophenone-8, such as marketed under the trademark “Spectra-Sorb UV-24” by American Cyanamid; benzophenone-9 (Disodium dihydroxy dimethoxy benzophenonedisulfonate), such as marketed under the trademark “Uvinul DS-49” by BASF; and benzophenone-12, and n-hexyl 2-(4-diethylamino-2-hydroxybenzoyl)benzoate (such as UVINUL A+ by BASF). β, β-Diphenylacrylate compounds: Octocrylene, such as marketed in particular under the trademark “Uvinul N539” by BASF; and Etocrylene, such as marketed in particular under the trademark “Uvinul N35” by BASF. Triazine compounds: Diethylhexyl butamido triazone, such as marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as marketed under the trademark «TINOSORB S» by CIBA GEIGY, and ethylhexyl triazone, such as marketed under the trademark «UVTNUL T150>> by BASF. Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975. Benzalmalonate compounds: Dineopentyl 4′-methoxybenzalmalonate, and polyorganosiloxane comprising benzalmalonate functional groups, such as polysilicone-15, such as marketed under the trademark “Parsol SLX” by Hoffmann-LaRoche. Benzimidazole compounds, in particular, phenylbenzimidazole derivatives: Phenylbenzimidazole sulfonic acid, such as marketed in particular under the trademark “Eusolex 232” by Merck, and disodium phenyl dibenzimidazole tetrasulfonate, such as marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264.

Para-aminobenzoic acid compounds: PABA (p-aminobenzoic acid), ethyl PABA, Ethyl dihydroxypropyl PABA, pentyl dimethyl PABA, ethylhexyl dimethyl PABA, such as marketed in particular under the trademark “Escalol 507” by ISP, glyceryl PABA, and PEG-25 PABA, such as marketed under the trademark “Uvinul P25” by BASF. Methylene bis-(hydroxyphenylbenzotriazol) compounds, such as 2,2′-methylenebis[6-(2H-benzotriazol-2-yl)-4-methyl-phenol], such as marketed in the solid form under the trademark “Mixxim BB/200” by Fairmount Chemical, 2,2′-methylenebis[6-(2H-benzotriazol-2-yl)-4-(I,I,3,3-tetramethylbutyl)phenol], such as marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197, 26, 184, and EP-893,119, and Drometrizole trisiloxane, such as marketed under the trademark “Silatrizole” by Rhodia Chimie or- “Mexoryl XL” by L'Oréal. Benzoxazole compounds: 2,4-bis[5-I(dimethylpropyl)benzoxazol-2-yl-(4-phenyl)imino]- 6-(2-ethylhexyl)imino-1,3,5-triazine, such as marketed under the trademark of Uvasorb K2A by Sigma 3V. Screening polymers and screening silicones: The silicones described in WO 93/04665. Dimers derived from a-alkylstyrene: The dimers described in DE-19855649. 4,4-Diarylbutadiene compounds: 1,1-dicarboxy(2,2′-dimethylpropyl)-4,4-diphenylbutadiene.

It is in some embodiments desirable that the organic UV filter(s) be selected from the group consisting of: butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, 1,r-(1,4-piperazinediAbis[14244-(diethylamino)-2-hydroxybenzoyl]phenylFmethanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)- s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]- disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, 1,1-dicarboxy(2,2′-dimethylpropyI)-4,4-diphenylbutadiene, 2,4-bis[5-1 (dimethylpropyl) benzoxazol-2-yl-(4-phenyl)imino]-6-(2-ethylhexyl)imino-1,3,5-triazine, camphor benzylkonium methosulfate, and mixtures thereof.

In a preferred embodiment, the cosmetic composition of the present invention has UV filters in an amount ranging from about 0.1% to about 50% by weight, preferably in an amount of from about 5% to about 40% by weight, more preferably about 5% to about 30% by weight, most preferably about 10% to about 30% by weight, based on the total weight of the composition.

Pigments

The suitable pigments used in the cosmetic sunscreen composition of the present invention are uncoated or coated pigments. coated or uncoated metal oxide pigments (mean size of the primary particles: generally between 5 nm and 100 nm, preferably between 10 nm and 50 nm), for instance titanium oxide (amorphous or crystallized in rutile and/or anatase form), iron oxide, zinc oxide, zirconium oxide or cerium oxide pigments, which are all UV-photoprotective agents that are well known per se.

The pigments may or may not be coated.

The coated pigments are pigments that have undergone one or more surface treatments of chemical, electronic, mechanochemical and/or mechanical nature with compounds as described, for example, in Cosmetics & Toiletries, February 1990, Vol. 105, pages 53-64, such as amino acids, beeswax, fatty acids, fatty alcohols, anionic surfactants, lecithins, sodium, potassium, zinc, iron or aluminium salts of fatty acids, metal alkoxides (of titanium or aluminium), polyethylene, silicones, proteins (collagen, elastin), alkanolamines, silicon oxides, metal oxides or sodium hexametaphosphate.

As is known, silicones are organosilicon polymers or oligomers comprising a linear or cyclic and branched or crosslinked structure, of variable molecular weight, obtained by polymerization and/or polycondensation of suitably functionalized silanes and essentially constituted of a repetition of main units in which the silicon atoms are connected to each other via oxygen atoms (siloxane bond), optionally substituted hydrocarbon-based radicals being connected directly to said silicon atoms via a carbon atom.

The term “silicones” also encompasses the silanes required for their preparation, in particular alkylsilanes.

The silicones used for coating the pigments that are suitable for the present invention are preferably chosen from the group containing alkylsilanes, polydialkylsiloxanes and polyalkylhydrogenosiloxanes. Even more preferentially, the silicones are chosen from the group containing octyltrimethylsilane, polydimethylsiloxanes and polymethylhydrosiloxanes.

Needless to say, before being treated with silicones, the metal oxide pigments may have been treated with other surface agents, in particular with cerium oxide, alumina, silica, aluminium compounds or silicon compounds, or mixtures thereof.

The coated pigments are more particularly titanium oxides that have been coated:
with silica, such as the product Sunveil from the company Ikeda,
with silica and iron oxide, such as the product Sunveil F from the company Ikeda,
with silica and alumina, such as the products Microtitanium Dioxide MT 500 SA and Microtitanium Dioxide MT 100 SA from the company Tayca and Tioveil from the company Tioxide,
with alumina, such as the products Tipaque TTO-55 (B) and Tipaque TTO-55 (A) from the company Ishihara and UVT 14/4 from the company Kemira,
with alumina and aluminium stearate, such as the products Microtitanium Dioxide MT 100 T, MT 100 TX, MT 100 Z and MT-01 from the company Tayca, the products Solaveil CT-10 W and Solaveil CT 100 from the company Uniqema and the product Eusolex T-AVO from the company Merck,
with silica, alumina and alginic acid, such as the product MT-100 AQ from the company Tayca,
with alumina and aluminium laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR 351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminium stearate and treated with a silicone, such as the product STT-30-DS from the company Titan Kogyo,
with silica and treated with a silicone, such as the product UV-Titan X 195 from the company Kemira,
with alumina and treated with a silicone, such as the products Tipaque TTO-55 (S) from the company Ishihara or UV Titan M 262 from the company Kemira,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca;
TiO₂ treated with octyltrimethylsilane, sold under the trade name T 805 by the company Degussa Silices;
TiO₂ treated with a polydimethylsiloxane, sold under the trade name 70250 Cardre UF TiO₂Sl₃ by the company Cardre; and
anatase/rutile TiO₂ treated with a polydimethylhydrogenosiloxane, sold under the trade name Microtitanium Dioxide USP Grade Hydrophobic by the company Color Techniques.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wackher under the name Transparent titanium oxide PW, by the company Miyoshi Kasei under the name UFTR, by the company Tomen under the name ITS and by the company Tioxide under the name Tioveil AQ.

The uncoated zinc oxide pigments are, for example:
those sold under the name Z-Cote by the company Sunsmart,
those sold under the name Nanox by the company Elementis,
those sold under the name Nanogard WCD 2025 by the company Nanophase Technologies.

The coated zinc oxide pigments are, for example:
those sold under the name Zinc Oxide CS-5 by the company Toshibi (ZnO coated with polymethylhydrosiloxane),
those sold under the name Nanogard Zinc Oxide FN by the company Nanophase Technologies (as a 40% dispersion in Finsolv TN, C₁₂-C₁₅ alkyl benzoate),
those sold under the name Daitopersion ZN-30 and Daitopersion ZN-50 by the company Daito (dispersions in cyclopolymethylsiloxane/oxyethylenated polydimethylsiloxane, containing 30% or 50% of nano zinc oxides coated with silica and polymethylhydrosiloxane),
those sold under the name NFD Ultrafine ZnO by the company Daikin (ZnO coated with perfluoroalkyl phosphate and copolymer based on perfluoroalkylethyl as a dispersion in cyclopentasiloxane),
those sold under the name SPD-Z1 by the company Shin-Etsu (ZnO coated with silicone-grafted acrylic polymer, dispersed in cyclodimethylsiloxane),
those sold under the name Escalol Z100 by the company ISP (alumina-treated ZnO dispersed in an ethylhexyl methoxycinnamate/PVP-hexadecene/methicone copolymer mixture),
those sold under the name Fuji ZnO-SMS-10 by the company Fuji Pigment (ZnO coated with silica and polymethylsilsesquioxane),
those sold under the name Nanox Gel TN by the company Elementis (ZnO dispersed at a concentration of 55% in C₁₂-C₁₅ alkyl benzoate with hydroxystearic acid polycondensate).

The uncoated cerium oxide pigments are sold under the name Colloidal Cerium Oxide by the company Rhone-Poulenc.

The uncoated iron oxide pigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220.

The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.

Mention may also be made of mixtures of metal oxides, in particular of titanium dioxide and of cerium dioxide, including the equal-weight mixture of titanium dioxide and cerium dioxide coated with silica, sold by the company Ikeda under the name Sunveil A, and also the mixture of titanium dioxide and zinc dioxide coated with alumina, silica and silicone, such as the product M 261 sold by the company Kemira, or coated with alumina, silica and glycerol, such as the product M 211 sold by the company Kemira.

In a preferred embodiment, the pigments used in the cosmetic composition of the present invention are selected from the group consisting of metal oxide pigments, such as titanium dioxide, zinc oxide, titanium oxide, iron oxide, zirconium oxide and cerium oxide, or mixtures thereof.

The pigments may be present in the compositions according to the invention in a concentration of between 0.1% and 10% and preferably between 0.5% and 8% by weight, more preferably about 1.5% to about 7.0% by weight, most preferably about 2.5% to about 6.5% by weight relative to the total weight of the composition.

Additional Ingredients

In addition to the essential components described hereinbefore, the composition of the invention may further comprise any usual cosmetically acceptable ingredient, which may be chosen especially from such as inorganic UV filters, coated pigments, perfume/fragrance, preserving agents, solvents, actives, fatty compounds, vitamins, fillers, silicones, polymers, and mixtures thereof.

A person skilled in the art will take care to select the optional additional ingredients and/or the amount thereof such that the advantageous properties of the composition according to the invention are not, or are not substantially, adversely affected by the envisaged addition.

Suitable coated pigments are more particularly titanium oxides coated with silica such as the product, silica and iron oxide, silica and alumina, alumina such as the products, alumina and aluminum stearate, alumina and aluminum laurate, iron oxide and iron stearate, zinc oxide and zinc stearate, silica, alumina and silicone, silica, alumina, aluminum stearate and silicone, alumina and silicone, etc.

Mixtures of metal oxides may also be mentioned, especially titanium dioxide and cerium dioxide, including the silica-coated equiponderous mixture of titanium dioxide and cerium dioxide, as well as the alumina-silica- and silicone-coated mixture of titanium oxide and zinc dioxide, or the alumina-, silica- and glycerin-coated mixture of titanium dioxide and zinc dioxide.

Suitable polymers include, but are not limited to, aluminum starch octenylsuccinate, xanthan gum, poly 010-30 alkyl acrylate, acrylates/C₀₋₃₀ alkyl acrylate crosspolymer, styrene/acrylates copolymer, and mixtures thereof.

The composition may also comprise at least one silicon ingredient, which may be dimethicone and caprylyl methicone, among others.

Non-limiting example of preserving agent which can be used in accordance with the invention include phenoxyethanol.

Suitable solvents include, but are not limited to water, alcohols, glycols and polyols such as glycerin, water, caprylyl glycol, pentylene glycol, propylene glycol, butylene glycol, C₁₂₋₁₅ alkyl benzoate and mixtures thereof.

Suitable additional actives include, but are not limited to, disodium EDTA, triethanolamine, and mixtures thereof.

Exemplary of fat or oil materials include, but are not limited to, esters, fatty acids, synthetic oils, and hydrocarbons/paraffins, such as steelyl alcohol, myristic acid, palmitic acid. silicones mineral oil, plant/vegetable oils, and mixtures thereof.

Non-limiting example of vitamins suitable for the composition of the present invention includes tocopherol.

Examples of silicones used in the composition of the present invention but not limited to are dimethicone and caprylyl methicone.

Exemplary of polymers, include, but not limited to, aluminum starch octenylsuccinate, xanthan gam, acrylates/C₁₀₋₃₀ alkyl acrylate crosspolymer and styrene/acrylates copolymer.

The cosmetic composition may contain, for example, at least one carbon-based oil, hydrocarbon-based oil, fluorooil and/or silicone oil of mineral, plant or synthetic origin.

The term “hydrocarbon-based oil” means oils mainly containing carbon atoms and hydrogen atoms and in particular alkyl or alkenyl chains, for instance alkanes or alkenes, but also oils with an alkyl or alkenyl chain comprising one or more alcohol, ether, ester and/or carboxylic acid groups.

As oils that may be used, mention may thus be made, this list not being limiting, of hydrocarbon-based oils of mineral or synthetic origin such as linear or branched hydrocarbons, for instance liquid paraffin and its derivatives, liquid petroleum jelly, polydecenes, hydrogenated polyisobutene such as Parleam sold by Nippon Oil Fats, squalane of synthetic or plant origin; oils of animal origin, such as mink oil, turtle oil or perhydrosqualene; hydro-carbon-based oils of plant origin with a high triglyceride content consisting of fatty acid esters of glycerol, the fatty acids of which may have varied chain lengths, said chains possibly being linear or branched, and saturated or unsaturated, for instance sweet almond oil, beauty-leaf oil, palm oil, grapeseed oil, sesame oil, arara oil, rapeseed oil, sunflower oil, cottonseed oil, apricot oil, castor oil, alfalfa oil, marrow oil, blackcurrant oil, macadamia oil, musk rose oil, hazelnut oil, avocado oil, jojoba oil, olive oil or cereal germ oil (from corn, wheat, barley or rye); fatty acid esters and especially esters of lanolic acid, of oleic acid, of lauric acid or of stearic acid; synthetic esters such as, for example, purcellin oil (cetostearyl octanoate), isononyl isononanoate, C₁₂ to C₁₅ alkyl benzoate, 2-ethylhexyl palmitate, octanoates, decanoates or ricinoleates of alcohols or of polyalcohols, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyl-dodecyl myristate, 2-diethylhexyl succinate, diiso-stearyl malate, or glyceryl or diglyceryl triiso-stearate; hydroxylated esters, for instance isostearyl lactate; pentaerythritol esters; C₈-C₂₆ higher fatty acids such as oleic acid, linoleic acid, linolenic acid or isostearic acid; C8-C26 higher fatty alcohols such as oleyl alcohol, linoleyl alcohol, linolenyl alcohol, isostearyl alcohol or octyldodecanol; synthetic esters containing at least 7 carbon atoms, silicone oils such as polydimethylsiloxanes (PDMSs) that are liquid at room temperature, linear, and optionally phenylated, such as phenyltrimethicones, phenyltrimethylsiloxydi-phenylsiloxanes, diphenyldimethicones, diphenylmethyl-diphenyltrisiloxanes, liquid 2-phenylethyl trimethyl-siloxysilicates, optionally substituted with aliphatic and/or aromatic groups, for instance alkyl, alkoxy or phenyl groups that are pendent and/or at the end of a silicone chain, these groups containing from 2 to 24 carbon atoms and being optionally fluorinated, or with functional groups such as hydroxyl, thiol and/or amine groups; polysiloxanes modified with fatty acids, with fatty alcohols or with polyoxyalkylenes, for instance dimethicone copolyols or alkylmethicone copolyols; liquid fluorosilicones; or caprylic/capric acid triglycerides, for instance those sold by Stearineries Dubois or those sold under the names Miglyol 810, 812 and 818 by Dynamit Nobel; and mixtures thereof.

The additional ingredients may represent from 1% to 85%, such as from 10% to 80% or such as from 25 to 75% by weight of the total weight of the composition of the invention.

By way of non-limiting illustration, the invention will now be described with reference to the following examples.

### EXAMPLES

### Examples 1 to 5

Suitable compositions according to the present invention are as Examples 1 to 3, and Comparative Examples are 4 to 6, as follows:

| | | EX. 1 | EX. 2 | EX. 3 | EX. 4 | EX. 5 | EX. 6 |
|---|---|---|---|---|---|---|---|
| FUNCTION | INGREDIENT | (%) | (%) | (%) | (%) | (%) | (%) |
| fatty | Copernicia cerifera | 1.5 | 1.5 | 1.5 | 1.5 | 1.5 | 1.5 |
| compound | (carnauba) wax | | | | | | |
| | isononyl isononanoate | 5 | 5 | 5 | 5 | 5 | 5 |
| polymer | polyamide-8 | 1.5 | 1.5 | 1.5 | 1.5 | — | 1.5 |
| | hydroxyethyl acrylate/sodium | 0.5 | 0.5 | 0.5 | 0.5 | — | 0.5 |
| | acryloyldimethyl taurate | | | | | | |
| | copolymer | | | | | | |
| | polyacrylate crosspolymer-6 | — | — | — | — | 0.5 | — |
| UV filter | ethylhexyl salicylate | 20.5 | 30.5 | — | 30.5 | 20.5 | 20 |
| | homosalate | | | | | | |
| | titanium dioxide | | | | | | |
| | ethylhexyl triazone | | | | | | |
| | bis-ethylhexyloxyphenol | | | | | | |
| | methoxyphenyl triazine | | | | | | |
| | terephthalylidene dicamphor | | | | | | |
| | sulfonic acid | | | | | | |
| | butyl | | | | | | |
| | methoxydibenzoylmethane | | | | | | |
| | phenylbenzimidazole | | | | | | |
| | sulfonic acid | | | | | | |
| surfactant | PEG-30 | 2.5 | 2.5 | 2.5 | — | — | 2.5 |
| | dipolyhydroxystearate | | | | | | |
| | octyldodecanol (and) | 3.0 | 3.0 | 3.0 | 3.0 | 3.0 | 3.0 |
| | octyldodecyl xyloside | | | | | | |
| filler | silica | 6 | 6 | 6 | 4 | — | 6 |
| | silica silylate | 2.5 | 2.5 | 2.5 | 2.5 | 2.5 | 2.5 |
| additional | cetyl PEG/PPG-10/1 | 4 | 3 | 4 | 4 | 3 | 3 |
| ingredients | dimethicone | | | | | | |
| | disodium EDTA | | | | | | |
| | triethanolamine | | | | | | |
| | phenoxyethanol | | | | | | |
| dye/pigments | iron oxides (and) | — | 8 | — | — | — | 8 |
| | perfluorohexylethyl | | | | | | |
| | triethoxysilane | | | | | | |
| | iron oxides (and) | | | | | | |
| | perfluorohexylethyl | | | | | | |
| | triethoxysilane | | | | | | |
| | titanium dioxide (and) | | | | | | |
| | aluminum hydroxide (and) | | | | | | |
| | perfluorohexylethyl | | | | | | |
| | triethoxysilane | | | | | | |
| | iron oxides (and) | | | | | | |
| | perfluorohexylethyl | | | | | | |
| | triethoxysilane | | | | | | |
| | iron oxides | | | | | | |
| | iron oxides | | | | | | |
| | iron oxides | | | | | | |
| | titanium dioxide | | | | | | |
| solvent | caprylyl glycol | 1 | 1 | 1 | 1 | 1 | 1 |
| | isododecane | 14.5 | 14.5 | 14.5 | 14.5 | 14.5 | 14.5 |
| | glycerin | 2.5 | 2.5 | 2.5 | 2.5 | 2.5 | 2.5 |
| | water | QSP 100 | QSP 100 | QSP 100 | QSP 100 | QSP 100 | QSP 100 |

### Example 6

The following Example is another embodiment of the present composition, as follows:

| FUNCTION | INGREDIENT | EX. 6 (%) |
|---|---|---|
| FATTY | COPERNICIA CERIFERA (CARNAUBA) WAX | 2 |
| COMPOUND | ISONONYL ISONONANOATE | 5 |
| POLYMER | POLYAMIDE-8 | 1.5 |
| | HYDROXYETHYL ACRYLATE/SODIUM | 0.5 |
| | ACRYLOYLDIMETHYL TAURATE COPOLYMER | |
| | POLYACRYLATE CROSSPOLYMER-6 | — |
| UV FILTER | ETHYLHEXYL SALICYLATE | 24 |
| | HOMOSALATE | |
| | TITANIUM DIOXIDE | |
| | ETHYLHEXYL TRIAZONE | |
| | BIS-ETHYLHEXYLOXYPHENOL | |
| | METHOXYPHENYL TRIAZINE | |
| | TEREPHTHALYLIDENE DICAMPHOR SULFONIC | |
| | ACID | |
| | BUTYL METHOXYDIBENZOYLMETHANE | |
| | PHENYLBENZIMIDAZOLE SULFONIC ACID | |
| | OCTOCRYLENE | |
| SURFACTANT | PEG-30 DIPOLYHYDROXYSTEARATE | 2.5 |
| | OCTYLDODECANOL (and) OCTYLDODECYL | 3.0 |
| | XYLOSIDE | |
| FILLER | SILICA | 6 |
| | SILICA SILYLATE | 2.0 |
| | KAOLIN | 0.4 |
| ADDITIONAL | CETYL PEG/PPG-10/1 DIMETHICONE | 4 |
| INGREDIENTS | DISODIUM EDTA | |
| | TRIETHANOLAMINE | |
| | PHENOXYETHANOL | |
| DYE/PIGMENTS | IRON OXIDES (and) PERFLUOROHEXYLETHYL | — |
| | TRIETHOXYSILANE | |
| | IRON OXIDES (and) PERFLUOROHEXYLETHYL | |
| | TRIETHOXYSILANE | |
| | TITANIUM DIOXIDE (and) ALUMINUM HYDROXIDE | |
| | (and) PERFLUOROHEXYLETHYL | |
| | TRIETHOXYSILANE | |
| | IRON OXIDES (and) PERFLUOROHEXYLETHYL | |
| | TRIETHOXYSILANE | |
| | IRON OXIDES | |
| | IRON OXIDES | |
| | IRON OXIDES | |
| | TITANIUM DIOXIDE | |
| FRAGRANCE | FRAGRANCE | 0.1 |
| SOLVENT | CAPRYLYL GLYCOL | 1 |
| | ISODODECANE | 13.5 |
| | GLYCERIN | 2.5 |
| | WATER | QSP 100 |

### Examples 7 and 8

Examples 7 and 8 are examples of the state of the art:

| Ex. 7 | Ex. 8 |
|---|---|
| TEREPHTHALYLIDENE DICAMPHOR | Water |
| SULFONIC ACID | |
| GLYCERYL STEARATE (and) PEG-100 | ETHYLEHEXYL |
| STEARATE | METHOXYCINNAMATE |
| STEARYL ALCOHOL | BUTYLENE GLYCOL |
| WATER | PROPYLENE GLYCOL |
| ALUMINUM STARCH | POLYMETHYL METACRYLATE |
| OCTENYLSUCCINATE | |
| TOCOPHEROL | DIMETHICONE |
| BUTYL METHOXYDIBENZOYLMETHANE | PEG-8 |
| METHYLENE BIS-BENZOTRIAZOLYL | BUTYL |
| TETRAMETHYLBUTYLPHENOL (and) | METHOXYDIBENZOYLMETHANE |
| POLYGLYCERYL-10 LAURATE | |
| PHENYLBENZIMIDAZOLE SULFONIC | ETHYLHEXYL TRIAZONE |
| ACID | |
| AMMONIUM | SILICA |
| ACRYLOYLDIMETHYLTAURATE/VP | |
| COPOLYMER | |
| TITANIUM DIOXIDE | PHENOXYETHANOL |
| CAPRYLYL GLYCOL | TITANIUM DIOXIDE |
| DISODIUM EDTA | ACRYLATES/C10-30 ALKYL |
| | ACRYLATE CROSSPOLYMER |
| SILICA SILYLATE | TOCOPHERYL ACETATE |
| PENTYLENE GLYCOL | ALLANTOIN |
| CAPRYLYL METHICONE | ETHYLHEXYLGLYCERIN |
| FRAGRANCE | PARFUM (FRAGRANCE) |
| LAURYL METHACRYLATE/GLYCOL | SODIUM HYALURONATE |
| DIMETHACRYLATE CROSSPOLYMER | |
| TRIETHANOLAMINE | SODIUM HYDROXIDE |
| CETYL PEG/PPG-10/1 DIMETHICONE | CARBOMER |
| DISODIUM EDTA | DISODIUM EDTA |
| TRIETHANOLAMINE | TOCOPHEROL |
| PHENOXYETHANOL | BHT |
| STEARIC ACID | ASCORBYL PALMITATE |
| POTASSIUM CETYL PHOSPHATE | ASCORBIC ACID |
| OCTOCRYLENE | CITRIC ACID |
| SILICA | |
| SODIUM METHYL STEAROYL TAURATE | |
| BIS-ETHYLHEXYLOXYPHENOL | |
| METHOXYPHENYL TRIAZINE | |
| ETHYLHEXYL TRIAZONE | |
| GLYCERIN | |
| DIISOPROPYL SEBACATE | |
| HOMOSALATE | |
| POLOXAMER 338 | |
| ISONONYL ISONONANOATE | |
| SILICA (and) TITANIUM DIOXIDE | |
| ACRYLATES/C10-30 ALKYL ACRYLATE | |
| CROSSPOLYMER | |

The compositions Ex. 1 to 3, according to the present invention, and compositions Ex. 7 and 8, comparative examples, were used in a consumer test in order to provide performance results regarding evidence of pores, smooth/soft touch, look throughout the assessment, matte effect and shine control, of the compositions of the present invention in view of state of the art compositions.

The compositions according to the present invention, Ex. 1 to 3, demonstrated improved effects when compared to the naked skin and to benchmark compositions.

Compositions Examples 1 to 3 demonstrated benefits over Ex. 7, regarding pores less evident, smooth/soft touch and better look throughout the assessment.

Compositions Examples 1 to 3 demonstrated benefits over Ex. 8, regarding matte effect, soft focus (pores less evident) and with more shine control.

When comparing the Compositions Examples 1 to 3 over the naked skin, the benefits were noticed regarding dry touch, matte effect, soft touch and smooth skin with a more homogenous look.

### Examples 9 and 10

Examples 9 and 10 below were used in a comparative test for the evaluation of sebum shininess control of the compositions, wherein Ex. 9 Ex. 10 are compositions according to the invention.

| Ex. 9 | Ex. 10 |
|---|---|
| CETEARYL DIMETHICONE | SILICA |
| CROSSPOLYMER | |
| CETEARYL ALCOHOL | PHENYLBENZIMIDAZOLE SULFONIC |
| | ACID |
| WATER | BIS-ETHYLEXYLOXYPHENOL |
| | METHOXYPHENY TRIAZINE |
| ALUMINUM STARCH | ETHYLHEXYL TRIAZONE |
| OCTENYLSUCCINATE | |
| CETEARYL GLUCOSIDE | HOMOSALATE |
| BUTYL METHOXYDIBENZOYLMETHANE | GLYCERIN |
| METHYLENE BIS-BENZOTRIAZOLYL | DIMETHICONE |
| TETRAMETHYLBUTYLPHENOL | |
| PHENYLBENZIMIDAZOLE SULFONIC | ALUMINUM STARCH |
| ACID | OCTENYLSUCCINATE |
| TITANIUM DIOXIDE | WATER |
| DISODIUM EDTA | DIISOPROPYL SEBACATE |
| PHENOXYETHANOL | GLYCINE SEED EXTRACT |
| CAPRYLYL METHICONE | OCTOCRYLENE |
| CETYL PALMITATE | BUTYL-METHOXYDIBENZOILMETHANE |
| DIMETHICONE | DISSODIUM PHENYL DIBENZIMIDAZOLE |
| | TETRASULFONATE |
| DISODIUM PHENYL DIBENZIMIDAZOLE | ISONONYL ISONONANOATE |
| TETRASULFONATE | |
| OCTOCRYLENE | METHYLENE BIS-BENZOTRIAZOLYL |
| | TETRAMETHYLBUTYLPHENOL |
| SILICA | TRACONTANYL PVP |
| BIS-ETHYLHEXYLOXYPHENOL | CETEARYL ALCOHOL |
| METHOXYPHENYL TRIAZINE | |
| ETHYLHEXYL TRIAZONE | DIISOPROPYL ADIPATE |
| GLYCERIN | CAPRYLYL METHICONE |
| DIISOPROPYL SEBACATE | NYLON-12 |
| HOMOSALATE | ETHYLHEXYLGLYCERIN |
| ISONONYL ISONONANOATE | SODIUM HYDROXIDE |
| ACRYLATES/C10-30 ALKYL ACRYLATE | BENZYL ALCOHOL |
| CROSSPOLYMER | |
| GLYCINE SOJA SEED EXTRACT | INULIN LAURYL CARBAMATE |
| TRACONTANYL PVP | TITANIUM DIOXIDE |
| DIISOPROPYL ADIPATE | PHENOXYETHANOL |
| NYLON-12 | CETYL PALMITATE |
| ETHYLHEXYLGLICERIN | CETEARYL DIMETHICONE |
| | CROSSPOLYMER |
| SODIUM HYDROXIDE | CETEARYL GLUCOSIDE |
| BENZYL ALCOHOL | HYDROXYETHYL ACRYLATE/SODIUM |
| | ACRYLOYLDIMETHYL TAURATE |
| | COPOLYMER |
| INULIN LAURYL CARBAMATE | ACRYLATES/C10-C30 ALKYL |
| | ACRYLATE CROSSPOLYMER |
| HYDROXYETHYL ACRYLATE/SODIUM | CAPRYLOYL GLYCINE |
| ACRYLOYLDIMETHYL TAURATE | |
| COPOLYMER | |
| ISOHEXADECANE | DISSODIUM EDTA |
| CAPRYLOYIL GLYCINE | ISOHEXADECANE |
| TOCOPHERYL ACETATE | TOCOPHERYL ACETATE |
| SARCOSINE | SARCOSINE |
| POLYSORBATE 60 | POLYSORBATE 60 |
| CINNAMOMUM ZEYLANICUM BARK | CINNAMOMUM ZEYLANICUM BARK |
| EXTRACT | EXTRACT |

The test was to assess and compare the effect on shininess controls of Ex. 9 and 10, until 2 hours of exposure in Hammam room conditions (37°+/−1° C.+/−5% RH).

The volunteers arrived without product on the face and stayed in a controlled environment for 20 min (21° C.±1; 45%±5 RH) before the first measurements (T0-baseline values), when sebum and shininess levels were evaluated using the equipment Sebumeter and LightCam. After that, the studied formulas were randomly applied on half face by the investigator. The volunteers stayed in controlled environment for 10 min (21° C.±1; 45%±5 RH). Thereafter, shininess and sebum levels were evaluated (T10 min). After 2 h in Hammam room conditions (T2H) the shininess and sebum measurements were repeated.

The formulas Ex. 9 and 10 statistically reduced sebum casual levels 10 minutes after application and after 2 hours in Hammam room conditions. In addition, both formulas significantly increased the sebum after 2 hours in Hammam room conditions in comparison to T10 min.

Formula Ex. 10 reduced sebum casual levels 10.98% more effectively than Ex. 9, 10 minutes after its application and no differences were observed after 2 hours in Hammam room conditions.

The formulas Ex. 9 and 10 statistically reduced shininess levels 10 minutes after application and after 2 hours in Hammam room conditions. In addition, both formulas significantly increased the shininess after 2 hours in Hammam room conditions in comparison to T10 min.

The formula Ex. 9 reduced shininess levels 19.21% more effectively than formula EX. 10, 10 minutes after application and 20.13% after 2 hours in Hammam room.

The formula Ex. 10 showed statistically significant decrease of sebum casual level 10 minutes after the application (p=0.000) and also after 2 hours in Hammam room (p=0.000) in comparison to the initial condition (TO). In addition, the sebum casual level after 2 hours in Hammam room showed a statistically significant increase in comparison to T10 min (p=0.000).

The formula Ex. 9 showed statistically significant decrease of sebum casual level 10 minutes after the application (p=0.000) and also after 2 hours in Hammam room (p=0.000) in comparison to the initial condition (TO). In addition, the sebum casual level after 2 hours in Hammam room showed a statistically significant increase in comparison to T10 min (p=0.017).

The formula Ex. 10 and formula Ex. 9 showed a statistically significant difference between them considering shininess level 10 minutes after the application (p=0.009) with the side treated with formula Ex. 10 presenting lower sebum values. The formula Ex. 10 reduced the shininess level 10.98% more than the formula Ex. 9.

No statistically significant differences (p=0.684) were observed between the formulas, 2 hours after Hammam room. The formula Ex. 10 presented an average reduction of 1.11% but as already explained, this difference was not statistically significant.

As an resulted, it was concluded that:

The formulas Ex. 10 and 9 statistically significantly reduced sebum casual levels 10 minutes after application and after 2 hours in Hammam room conditions. In addition, both formulas significantly increased the sebum after 2 hours in Hammam room conditions in comparison to T10 min.

The formula Ex. 10 reduced sebum casual levels 10.98% more effectively than formula b49053 10 minutes after its application and no differences were observed after 2 hours in Hammam room conditions.

The formulas Ex. 10 and 9 statistically significantly reduced shininess levels 10 minutes after application and after 2 hours in Hammam room conditions. In addition, both formulas significantly increased the shininess after 2 hours in Hammam room conditions in comparison to T10 min.

The formula Ex. 9 reduced shininess levels 19.21% more effectively than formula EX. 10, 10 minutes after application and 20.13% after 2 hours in Hammam room.

1. A cosmetic composition comprising:
(a) at least a polymer, selected from water-soluble or water dispersible AMPS copolymers;
(b) at least a polyamide, selected from the group of aliphatic polyamides;
(c) at least a filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch, and mixtures thereof;
(d) at least a fatty compound, selected from carnauba wax, isononyl isononanoate and mixtures thereof;
(e) at least a nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants, and mixtures thereof.

2. The cosmetic composition, according to claim 1, comprising from 0.5% to 10% by weight of the at least one polymer, including all ranges and sub-ranges there between, based on the total weight of the composition.

3. The cosmetic composition, according to claim 1, comprising from 1% to 10% by weight of the at least one polyamide, including all ranges and sub-ranges there between, based on the total weight of the composition.

4. The cosmetic composition, according to claim 1, wherein the polyamide is selected from the group of polyamide-4, polyamide-6, polyamide-8, polyamide-11, polyamide-12, polyamide-4,6, polyamide-6,6, polyamide-6,9, polyamide-6,10, polyamide-6,12, and mixtures thereof.

5. The cosmetic sunscreen composition, according to claim 1, comprising from 1% to 15% by weight of the at least one filler, including all ranges and sub-ranges there between, based on the total weight of the composition.

6. The cosmetic composition, according to claim 1, comprising from 1% to 12% by weight of at least one fatty compound, including all ranges and sub-ranges there between, based on the total weight of the composition.

7. The cosmetic composition, according to claim 1, comprising from 1% to 10% by weight of at least one nonionic surfactant, including all ranges and sub-ranges there between, based on the total weight of the composition.

8. The cosmetic composition, according to claim 1, wherein it further comprises UV filters.

9. The cosmetic composition, according to claim 1, wherein it further comprises additional ingredients selected from coated and/or uncoated pigments, perfume/fragrance, preserving agents, solvents, actives, fatty compounds, vitamins, fillers, silicones, polymers and mixtures thereof.

10. The cosmetic composition, according to claim 1, wherein it is a makeup composition, a cosmetic sunscreen composition, or a skin care composition.

11. A cosmetic composition, comprising:
(a) from 1 to 6% of at least one polymer, selected from a water-soluble or water dispersible AMPS copolymers;
(b) from 1% to 6% of at least a polyamide selected from the group of polyamide-4, polyamide-6, polyamide-8, polyamide-11, polyamide-12, polyamide-4,6, polyamide-6,6, polyamide-6, 9, and polyamide-6,10, polyamide-6,12, and mixtures thereof;
(c) from 1.5% to 8% of at least one filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch;
(d) from 1.5% to 10% of at least a fatty compound, selected from carnauba wax and isononyl isononanoate, and mixtures thereof;
(e) from 1.5% to 6% of at least one nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants.

12. The cosmetic composition, according to claim 11, wherein it further comprises UV filters and/or pigments.

13. Use of a cosmetic composition comprising:
(a) at least a polymer, selected from water-soluble or water dispersible AMPS copolymers;
(b) at least a polyamide, selected from the group of aliphatic polyamides;
(c) at least a filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch, and mixtures thereof;
(d) at least a fatty compound, selected from carnauba wax, isononyl isononanoate and mixtures thereof;
(e) at least a nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants, and mixtures thereof
wherein it is for the manufacture of a product to be used as cosmetic sunscreen daily product, a makeup product or a skin care product.

14. Use, according to claim 11, wherein the composition further comprises UV filters.

15. A process of manufacturing a cosmetic composition comprising:
(a) at least a polymer, selected from water-soluble or water dispersible AMPS copolymers;
(b) at least a polyamide, selected from the group of aliphatic polyamides;
(c) at least a filler, selected from the group comprising mica, silica, zea may (corn) starch, magnesium oxide, nylon-12, nylon-66, cellulose, polyethylene, talc, talc (and) methicone, talc (and) dimethicone, perlite, sodium silicate, pumice, PTFE, polymethyl methacrylate, Oryza sativa (rice) starch, aluminum starch octenylsuccinate, potato starch modified, alumina, silica silylate, calcium sodium borosilicate, magnesium carbonate, hydrated silica, dimethicone/vinyl dimethicone crosspolymer, sodium carboxylmethyl starch, and mixtures thereof;
(d) at least a fatty compound, selected from carnauba wax, isononyl isononanoate and mixtures thereof;
(e) at least a nonionic surfactant selected from the group consisting of: alkyl- and polyalkyl-esters of glycerol, mixtures of alkyl- and polyalkyl-esters of glycerol with polyglyceryl, oxyalkylenated fatty acid esters of glycerol; oxyalkylenated fatty acid esters of sorbitan; oxyalkylenated fatty acid esters; oxyalkylenated fatty alcohol ethers, sugar esters, fatty alcohols, fatty alcohol ethers of sugars and hydrocarbon-based or silicone surfactants, and mixtures thereof;
(f) optionally UV filters;
wherein the process comprises the following steps:
a) Heating the oily phase containing fatty compounds, emollients and polymer until 75° C.,
a1) when present in the cosmetic composition, adding and heating UV filters and pigments in step a);
b) Heating the water phase containing polymer and preservatives until 70° C.,
b1) when present in the composition, adding and heating UV filters in step b);
c) Adding the step (a) into step (b) homogenizing the mixture;
d) Adding the fillers and solvent below 45° C. homogenizing the mixture.
